(19) United States
(12) Patent Application Publication
(10) Pub. No.: US 2003/0235851 A1
(43) Pub. Date: Dec. 25, 2003

(54) Methods of using sense and/or nonsense suppression to make nucleic acid-peptide display libraries containing peptides with unnatural amino acid residues

(76) Inventors: Richard W. Roberts, South Pasadena, CA (US); Shuwei Li, Dallas, TX (US); Adam Frankel, Los Angeles, CA (US)

Correspondence Address: GRAY CARY WARE & FREIDENRICH LLP, 4365 EXECUTIVE DRIVE, SUITE 1100, SAN DIEGO, CA, 92121-2133, US

(21) Appl. No.: 10/418,751

(22) Filed: Apr. 18, 2003

Related U.S. Application Data
(60) Provisional application No. 60/373,901, filed on Apr. 19, 2002.

(51) Int. Cl. C12Q 1/68; G01N 33/53; C07K 9/00; C07K 14/00

(52) U.S. Cl. 435/6; 435/7.1; 530/322; 530/395

(57) ABSTRACT

Libraries of nucleic acid-peptide fusion molecules are provided, wherein the peptide component of the fusion molecules contains one or more unnatural amino acid residues. Methods of making such libraries using sense suppression and/or nonsense suppression also are provided, as are methods of screening such libraries to identify peptides having desirable characteristics. Accordingly, isolated nucleic acid-peptide molecules also are provided, wherein the peptide component has a desirable characteristic, and is composed partially or entirely of unnatural amino acid residues.

### RELATED APPLICATION DATA

This application claims the benefit of priority under 35 U.S.C. § 119(e) to U.S. application Ser. No. 60/373,901, filed Apr. 19, 2002, the entire content of which is incorporated herein by reference.

### GOVERNMENT SUPPORT

This invention was made in part with government support under Grant No. GM 60416 awarded by the National Institutes of Health. The government has certain rights in this invention.

### FIELD OF THE INVENTION

The present invention relates generally to nucleic acid-peptide display libraries, and more specifically to RNA display libraries, wherein peptides of the library contains unnatural amino acid residues, and to methods of making such libraries using tRNA sense suppression or nonsense suppression.

### BACKGROUND OF THE INVENTION

The ability to create libraries of peptides provides a large number of agents that can have desirable properties, particularly as drugs. Libraries of peptides can be randomly generated, or can be prepared based on a known peptide, wherein one or a few positions in the peptide, for example, antigen binding sites of an antibody, is varied. The peptides of a library can be examined, for example, using high throughput assay formats, to identify those peptides having a desirable property such as a high binding affinity for a target protein. As such, combinatorial peptide libraries are rich reservoirs for sieving novel ligands of many therapeutically interesting targets, including agonists or antagonists of receptors, epitopes of antibodies, and inhibitors of enzymes.

Since it is easier to determine a nucleotide sequence of a nucleic acid molecule encoding a peptide than it is to determine the amino acid sequence of a peptide, methods have been developed to prepare peptide libraries, wherein the encoding nucleic acid molecule remains physically associated with the peptide. Upon identifying a peptide having a desired activity, the sequence of the encoding nucleic acid associated with the peptide can be determined, thus providing information as to the amino acid sequence of the encoded peptide. Further, having the nucleic acid sequence readily allows the production of large amounts of the peptide, which can be prepared using routine cloning and expression methods, or by chemical synthesis. Such techniques, wherein a peptide is associated with its encoding nucleic acid, include those generating phage, ribosome, and mRNA display libraries. Since each peptides is physically associated with its own coding sequence, it is relatively easy to prepare large numbers of peptides that have been determined, by iterative cycles of selection, to have desired properties.

Great diversity can be generated in peptide libraries because any, or most any, of the twenty naturally occurring amino acids can be introduced at any particular site in a peptide. Nevertheless, the chemical diversity of a library is limited due to the availability of only twenty naturally occurring amino acids. Furthermore, the use only of the twenty naturally occurring amino acids is limited by the possibility that a peptide that is potentially useful as a therapeutic agent may be quickly degraded or otherwise inactivated upon administration to a patient due, for example, to proteases present in the patient. Similarly, infectious agents such as bacteria are more likely to develop resistance against peptides that contain only naturally occurring amino acids because enzymes that are produced by the bacteria and can inactivate a peptide drug are more likely to be active against peptides containing naturally occurring amino acids than they would be against a peptide containing an unnatural amino acid such as an amino acid analog or other non-naturally occurring amino acid residue.

Accordingly, it can be desirable to include unnatural amino acids in peptides, particularly with respect to libraries of peptides generated using combinatorial methods, because the diversity of the library can be substantially increased, and because peptides identified in such libraries as having a desirable property can be particularly useful for in vivo purposes. Unfortunately, while methods of making peptide libraries, wherein the peptides contain unnatural amino acids, have been described, no convenient methods have been described for generating such peptide libraries, wherein the peptide containing the unnatural amino acid is linked to its encoding nucleic acid. Thus, there exists a need for methods of generating libraries of peptides containing unnatural amino acids, wherein the peptide is linked to its encoding nucleic acid. The present invention satisfies this need and provides additional advantages.

### SUMMARY OF THE INVENTION

The present invention is based on a determination that unnatural (i.e., non-naturally occurring) amino acid residues can be introduced or incorporated into peptides of a library of nucleic acid-peptide fusion molecules. The introduction of unnatural amino acid residues into the peptide components of the library provides the advantage that it allows the generation of a library having substantially increased diversity for peptides of a given length beyond that obtained using only the twenty naturally occurring amino acids. A library of peptides containing unnatural amino acid residues provides the further advantage that, where the unnatural amino acid comprises, for example, a ligand for a particular receptor, enzyme, or the like, the incorporation of the ligand into the peptide provides a means to modify presentation of the ligand to its target molecule and, therefore, allows an identification of agents that can have particularly desirable agonist or antagonist activity or other desired characteristics. As such, the present invention allows the principles of in vitro evolution and selection to be applied to peptides, which can comprise a domain of a protein or an entire protein, wherein the amino acid residues incorporated therein extend beyond the 20 naturally occurring amino acids.

Accordingly, the present invention relates to a library of diverse nucleic acid-peptide fusion molecules, wherein each molecule of the library comprises an encoding nucleic acid translationally linked to an encoded peptide, and wherein one or more peptides of the library contain at least one unnatural amino acid residue. The encoding nucleic acid generally is a ribonucleic acid (RNA) such as an mRNA, which includes a coding sequence that can, but need not, be randomized, biased, or variegated in one or more positions, and can further include one or more translational regulatory elements. The encoding nucleic acid is translationally linked at or near its 3′ terminus to the C-terminus of its encoded peptide, i.e., is linked during the translation process due to the catalytic activity of a ribosomal peptidyl transferase. For example, an encoding nucleic acid can be translationally linked to its encoded peptide via a peptide acceptor such as puromycin, which can be bound to the 3′ terminus of the encoding nucleic acid or can be linked thereto via a linker molecule, for example, a deoxyribonucleic acid (DNA) or RNA linker. As such, the encoding nucleic acid, which comprises a coding sequence of the peptide, can further be a DNA-RNA hybrid, or can be a translatable RNA mimic, for example, RNA mimics containing 2′-O-methyl nucleotides or 2′-fluoro-nucleotides.

An unnatural amino acid can be any modified amino acid, including, for example, an amino acid analog or derivative, or can be an amino acid mimic. For example, the unnatural amino acid can be an amino acid analog such as biocytin; can be a peptoid monomer such as N-methyl glycine, N—(S)-phenylethyl glycine, or N-methyl-phenylalanine (see Example 4), or other N-substituted amino acid; can be an α-hydroxy acid, a hydrazino amino acid, an amino-oxy acid, a β-amino acid, a D-amino acid, or an achiral amino acid; or can be a naturally occurring amino acid that has been derivatized, for example, by modification of a functional group of the amino acid, particularly at the position of the amino acid side chain. For example, the amino acid functional group that is modified can be a thiol group, an amino group, a carboxyl group, a guanidinium group, a hydroxyl group, or a phenolic group; and the modified functional group can be a carboxylic acid, an acid halide, a carboxylic ester, a thioester, or a carbamate.

An unnatural amino acid, which can be post-translationally introduced or translationally incorporated into peptides of a library of the invention, also can be an amino acid residue comprising a ligand (or other specific binding pair member) or any other molecule of interest. For example, the ligand can be one that specifically binds a target molecule of interest, for example, an antibiotic, wherein the peptide comprising the antibiotic has greater or more specific affinity for a target molecule. A target molecule can be any molecule of interest, including, for example, a protein or a nucleic acid molecule, which can be a protein or nucleic acid molecule isolated from a prokaryotic or eukaryotic organism (or based on such a protein or nucleic acid molecule). For example, the target molecule can be an enzyme such as a bacterial enzyme, or a eukaryotic kinase or phosphatase; a protein involved in a signal transduction pathway (e.g., a G protein); or a cell surface receptor such as a hormone or cytokine receptor. The target molecule also can be a ligand for a receptor molecule; for example, the target molecule can be a peptide hormone or small organic molecule hormone, or can be a cytokine.

Also provided are nucleic acid-peptide fusion molecules selected from such a library, wherein the selected peptides include a peptide containing at least one unnatural amino acid residue, as well as isolated peptides cleaved from such fusion molecules. For example, a selected peptide can have an amino acid sequence as set forth in any of SEQ ID NOS: 5 to 18, each of which further comprises a conjugate of cysteine and 5-bromoacetyl penicillanic acid, which generates cysteine having a 6β-(2-methylsulfanyl-acetylamino)-penicillanic acid side group (beilstein registry number 38268, CAS Registry 91431-28-6; the cysteine conjugate hereinafter referred to as “cysteine-thioether-6-amido penicillanic acid”), or can be a peptide encoded by a nucleotide sequence as set forth in any of SEQ ID NOS: 37 to 45, wherein TAG is recognized by an orthogonal biocytin charged tRNA.

The present invention also relates to methods of producing libraries of diverse nucleic acid-peptide fusion molecules (also referred to as nucleic acid display libraries, RNA display libraries, or mRNA display libraries), wherein peptides of the library contain one or more unnatural amino acid residues. As disclosed herein, the unnatural amino acid can be introduced into peptides of the library post-translationally by modifying one or more amino acid residues, or can be introduced during translation using an appropriately charged orthogonal tRNA (e.g., THG73) or other appropriate tRNA (e.g., a tRNA based on an orthogonal tRNA such as the GUA, GCG, and GCU tRNAs; see Example 4). Accordingly, in one embodiment, the invention provides a method for producing a library of diverse nucleic acid-peptide fusion molecules, wherein each molecule of the library comprises an encoding nucleic acid translationally linked to an encoded peptide, and wherein at least one peptide of the library contains at least one unnatural amino acid residue. Such a method can be performed, for example, by in vitro translating peptide coding sequences of a plurality of RNA molecules, each RNA molecule having a 5′ end and a 3′ end, wherein each coding sequence of an RNA molecule is linked to a peptide acceptor at the 3′ end of the coding sequence, and wherein the peptide acceptor is translationally linked to a C-terminal amino acid residue of a growing peptide chain by a ribosomal peptidyl transferase; and contacting nucleic acid-peptide fusion molecules of the library with a peptide modifying agent under conditions suitable for post-translationally modifying at least one amino acid residue in at least one of the nucleic acid-peptide fusion molecules.

A peptide modifying agent can be a chemical reagent that modifies an amino acid residue in the peptide component of a nucleic acid-peptide fusion molecule, for example, a reagent that reacts with and modifies a functional group such as a reactive amino acid side chain; or can be an enzyme that catalytically modifies a functional group of an amino acid residue in peptides of the library. The peptide modifying agent is selected based on the particular functional group to be modified, as well as the resulting change desired, and is further selected on the basis of its being substantially inert to portions of the nucleic acid-peptide fusion molecule other than the functional group or groups to be modified. For example, it will be recognized that chemical reagents that are active under basic conditions generally are not useful for the present methods because RNA is susceptible to degradation under basic conditions.

A peptide modifying agent can modify a particular amino acid to generate the desired unnatural amino acid, or can modify the amino acid such that a second agent or reagent can be contacted with the amino acid to generate the desired unnatural amino acid. For example, a peptide modifying agent can be used to modify a thiol group, an amino group, a carboxyl group, a guanidinium group, a hydroxyl group, or a phenolic group of an amino acid, and can generate the desired unnatural amino acid, which comprises, for example, a carboxylic acid, an acid halide, a carboxylic ester, a thioester, a carbamate, a thiol group, an amino group, or a hydroxy group; or such a modified amino acid can be further contacted with a ligand or other moiety that has a reactive group than can react with the modified group on the amino acid, thus generating an unnatural amino acid comprising the ligand or other moiety. Such a ligand or moiety can be any molecule as desired, including a biologically active molecule, an affinity tag, a detectable label, a selectable marker, or other small organic molecule, peptide, protein, nucleic acid molecule, or the like.

A method of the invention can further include a step of isolating nucleic acid-peptide fusion molecules from the library. The nucleic acid-peptide fusion molecules can be isolated following post-translation modification to generate the unnatural amino acid residue or residues, or can be isolated prior to contacting the nucleic acid-peptide fusion molecules of the library with a peptide modifying agent, wherein the isolated fusion molecules then are post-translationally modified to generate the unnatural amino acid(s).

A method of the invention also can include, after contacting nucleic acid-peptide fusion molecules of the library with the peptide modifying agent, a step of further contacting the nucleic acid-peptide fusion molecules with a target molecule under conditions suitable for a specific interaction of the target molecule with peptides comprising an unnatural amino acid residue, for example, an unnatural amino acid comprising a ligand specific for the target molecule. Similarly, such a method can further include a step of isolating nucleic acid-peptide fusion molecules that specifically interact with the target molecule, and can further include isolating the peptide or the nucleic acid component of the fusion molecule.

A method of the invention is exemplified by introducing an unnatural amino acid residue comprising a conjugate of cysteine and 6-bromoacetyl penicillanic acid (cysteine-thioether-6-amido penicillanic acid), and selecting peptides that contain the unnatural amino acid and bind specifically to a penicillin binding protein. Such selected peptides are exemplified by the peptides having an amino acid sequence as set forth in SEQ ID NOS: 5 to 18, wherein the unnatural amino acid residue is a cysteine-thioether-6-amido penicillanic acid residue.

The invention also provides a library of diverse nucleic acid-peptide fusion molecules produced by such a method, wherein each molecule of the library comprises an encoding nucleic acid translationally linked to an encoded peptide, and wherein one or more peptides of the library contain at least one unnatural amino acid residue. In addition, a nucleic acid-peptide fusion molecule isolated from such a library is provided, including, for example, a nucleic acid-peptide fusion molecule selected following contact with a target molecule, wherein the peptide component of the fusion molecule, and particularly that portion or portions of the peptide comprising one or more unnatural amino acids, specifically interacts with the target molecule.

In another embodiment, a method for producing a library of diverse nucleic acid-peptide fusion molecules, wherein each molecule of the library comprises an encoding nucleic acid translationally linked to an encoded peptide, and wherein at least one peptide of the library contains at least one unnatural amino acid residue, is performed by contacting, under conditions suitable for in vitro translation of a peptide comprising an unnatural amino acid residue, a plurality of RNA molecules (e.g., mRNA molecules) comprising peptide coding sequences, wherein each RNA molecule has a 5′ end and a 3′ end, wherein each RNA molecule is linked at the 3′ end to a peptide acceptor, and wherein the peptide acceptor is translationally linked to a C-terminal amino acid residue of a growing protein chain by a ribosomal peptidyl transferase; and at least a first aminoacylated tRNA charged with a first unnatural amino acid residue, wherein one or more peptide coding sequences of the plurality of RNA molecules contain a codon specific for the first aminoacylated tRNA. An appropriate first (or other) aminoacylated tRNA can be an orthogonal tRNA charged with an unnatural amino acid residue or can be any other tRNA charged with an unnatural amino acid residue, provided the in vitro translation is performed under conditions in which editing of the aminoacylated tRNA is inhibited, for example, due to depletion aminoacyl tRNA synthases specific for the tRNA charged with the unnatural amino acid residue.

Such a method provides a means to incorporate one or more unnatural amino acid residues into one or more peptides by including, in addition to the first appropriate (e.g., orthogonal) aminoacylated tRNA, a second; a second and third; a second, third and fourth; etc., orthogonal aminoacylated tRNA molecules or other appropriate tRNA molecules, two or more of which can recognize different codons and be charged with different unnatural amino acid residues; or can recognize the same codon but be charged with different unnatural amino acid residues; or can recognize different codons but be charged with the same unnatural amino acid residue. As such, peptides translated according to this method can contain one, two, three or more different unnatural amino acid residues, or can be comprised entirely of unnatural amino acid residues.

An appropriate tRNA such as an orthogonal aminoacylated tRNA useful for purposes of the present invention can be any tRNA molecule that recognizes a particular codon (i.e., has a specific anti-codon) and is or can be charged with an unnatural amino acid. Preferably, the unnatural amino acid is not susceptible to removal from the orthogonal aminoacylated tRNA due to the editing/proof-reading function of an aminoacyl tRNA synthase. However, where the possibility exists that such editing can occur, the reaction can be further contacted with an aminoacyl tRNA synthetase inhibitor, for example, an aminoacyl sulfamide.

An appropriate tRNA such as an orthogonal aminoacylated tRNA can be any tRNA molecule that can be charged with an unnatural amino acid, including, for example, a suppressor tRNA, which recognizes a stop codon (e.g., an amber suppressor tRNA, which recognizes the stop codon, UAG), or a tRNA that is specific for a codon encoding an amino acid. The appropriate tRNA can be charged with an unnatural amino acid that is an analog or derivative of the amino acid normally encoded by the codon, or can be charged with any other unnatural amino acid residue. The unnatural amino acid can be any unnatural amino acid as described herein, including, for example, an unnatural amino acid comprising a ligand that specifically binds a target molecule, for example, a small organic molecule ligand such as an antibiotic, which can bind a bacterial enzyme, or a nucleoside, a nucleoside analog, a nucleotide, or a nucleotide analog, which can bind a signal transduction protein such as a G protein; a peptide ligand such as a peptide that acts as a substrate or cofactor for an enzyme (e.g., a kinase or phosphatase); or a polynucleotide ligand such as an oligonucleotide corresponding to a transcriptional regulatory element, which can bind a transcription factor.

As above, this embodiment of a method of the invention can further include a step of isolating nucleic acid-peptide fusion molecules of the library, wherein at least one peptide of the nucleic acid-peptide fusion molecules contains at least one unnatural amino acid residue; and/or can further include a step of contacting nucleic acid-peptide fusion molecules of the library with the target molecule under conditions suitable for a specific interaction of the target molecule with a ligand component of an unnatural amino acid residue, such a method which can further include isolating nucleic acid-peptide fusion molecules that specifically interact with the target molecule.

Accordingly, the present invention also relates to a library of diverse nucleic acid-peptide fusion molecules produced by such a method, which comprises sense and/or nonsense suppression, wherein each molecule of the library comprises an encoding nucleic acid translationally linked to an encoded peptide, and wherein at least one peptide of the library comprises at least one unnatural amino acid residue. Also provided is a population of nucleic acid-peptide fusion molecules isolated by the method of the invention, wherein at least one peptide of the nucleic acid-peptide fusion molecules comprises at least one unnatural amino acid residue; a population of peptides isolated from such a population of nucleic acid-peptide fusion molecules, wherein at least one peptide of the population of peptides comprises at least one unnatural amino acid residue; and a peptide isolated from such a population of nucleic acid-peptide fusion molecules, wherein the peptide comprises at least one unnatural amino acid residue. In addition, a population of nucleic acid-peptide fusion molecules isolated by a method of the invention is provided, wherein at least one peptide of the nucleic acid-peptide fusion molecules comprises at least one unnatural amino acid residue and specifically interacts with the target molecule; as is a population of peptides isolated from such nucleic acid-peptide fusion molecules, wherein at least one peptide of the population of peptides comprises at least one unnatural amino acid residue and specifically interacts with the target molecule; and a peptide isolated from such nucleic acid-peptide fusion molecules, wherein the peptide comprises at least one unnatural amino acid residue and specifically interacts with the target molecule.

### BRIEF DESCRIPTION OF THE FIGURES

FIG. 1A illustrates the construction of an mRNA display library containing an unnatural β-lactam side chain. The constant cysteine residue in peptide (SEQ ID NO: 4) generated in the mRNA fusion library is flanked by 5 randomized residues on both sides, and can react with 6-bromoacetyl penicillanic acid to form conjugates (cysteine-thioether-6-amido penicillanic acid; see Li and Roberts, Chem. Biol. 10:233-239, 2003, which is incorporated herein by reference).

FIG. 1B illustrates the selection cycle of the mRNA display library. The mRNA-display library, made from DNA template, can conjugate with a β-lactam through posttranslational modification. The resulting library containing the β-lactam side chain is subject to affinity selection against immobilized PBP2a. The enriched fraction with improved properties is amplified by PCR for the next cycle of selection.

FIG. 2 illustrates the specificity of cysteine modification. The library fusions containing a constant cysteine or control template fusions without cysteine, both labeled with ³⁵S-methionine, were reacted with 6-bromoacetyl penicillanic acid and incubated with immobilized β-lactamase mutant (E166A) for various times. The control fusions (open circles) did not bind to the matrix, whereas about 20% of the fusions containing cysteine (shaded circles) were bound to the matrix after 60 minutes. The sequences of the library (SEQ ID NO: 4) and control (SEQ ID NO: 46) fusions are shown.

FIG. 3 illustrates enrichment of selection. The lighter bars (to left) represent the percentage of ³⁵S-methionine labeled libraries arising from the 1^{st} cycle to the 9^{th} cycle bound to immobilized PBP2a resin. The dark bar (far right) represents the percentage of the library from the 9th cycle of selection bound to empty resin without PBP2a. The sequences of 15 clones from the library after 9 cycles of selection are shown (SEQ ID NOS: 5-18) below the sequence representing the library (SEQ ID NO: 4). Hyphen (-) represents the same residues as those in the first line (SEQ ID NO: 4), and dot (.) represents a deletion. A few cysteines appeared in randomized positions. The peptide shown as SEQ ID NO: 5 appeared twice.

FIG. 4 illustrates the relative inhibition curves of tested molecules (see Example 1). Symbols are as follows: diamond-cefotaxamine; solid circle-LRNSNC(pen)IRHFF (residues 2-12 of SEQ ID NO: 5); triangle-LRNSNC(COOH)IRHFF (residues 2-12 of SEQ ID NO: 5); open circle-EQKLIC(pen)SEEDL (SEQ ID NO: 19); and square-6-aminopenicillanic acid (6-APA).

FIG. 5A provides a scheme for insertion of unnatural amino acids into mRNA display libraries via amber suppression.

FIG. 5B shows the structure of biocytin-charged suppressor tRNA, THG73 (SEQ ID NO: 25).

FIG. 6 shows the sequences of nine nucleic acids in a library comprising peptides containing biocytin prior to selection (SEQ ID NOS: 28-36), and following one (SEQ ID NOS: 37-44; including two occurrences of SEQ ID NO: 37)or nine (SEQ ID NOS: 37, 44 and 45; including six occurrences, two occurrences, and one occurrence, respectively) cycles of selection against streptavidin agarose. Amino acid sequence (SEQ ID NO: 26) in three-letter format and encoding nucleotide sequence (SEQ ID NO: 27) of the library before selection is shown at the top (numbers refer to amino acid position). Xxx represents all 20 amino acid residue or UAG stop codon. NNS indicates randomized position—N is equal amount of all four nucleotides; S is 50% G plus 50% C. The sequences in the NNS saturation region and emerging UAG stop codons are shown. Residues identical to the original template are shown with a dash (-).

FIGS. 7A and 7B show a selection scheme for sense suppression.

FIG. 7A shows the exogenous biocytin-charged tRNA (SEQ ID NO: 25) and template library, including encoding nucleic acid (“cDNA”; SEQ ID NO: 52) and encoded peptide (SEQ ID NO: 53) sequences, used in the sense suppression translation reactions in rabbit reticulocyte lysate. The amber suppressor THG73 was mutated at the anticodon position to Watson-Crick base pair to all 16 GNN codons. The pool of 16 tRNAs were then acylated in one batch with the unnatural amino acid biocytin.

FIG. 7B illustrates that the incorporation of the unnatural amino acid into a mRNA-peptide fusion involves a competition between the endogenous tRNA population charged with natural amino acids (“aa”) and the exogenous tRNA pool charged with biocytin (“B”). The sequence present in the peptide is encoded in the covalently-attached mRNA through its 3′ puromycin (“P”), allowing the sequence information in the protein to be read and recovered via the attached RNA. By selecting for streptavidin binding through biocytin incorporation after several rounds of selection, the template library is enriched at the randomized position for GNN codons.

FIGS. 8A and 8B demonstrate that iterative selection rounds result in the enrichment of sense suppression by orthogonal tRNAs.

FIG. 8A shows the binding of (³⁵S)-methionine labeled mRNA-peptide fusions to streptavidin-agarose for rounds 0 to 4. Results are represented as percent counts per minute (CPM) of peptide bound to streptavidin as measured by scintillation counting. The minus sign (−) indicates the results of mRNA-peptide fusions generated without the addition of any exogenous tRNAs; the plus sign (+) indicates the results of fusions generated in the presence of 4.0 μg of the biocytin-tRNA^{NNC} pool in the translation reaction. For a higher stringency selection in round 3, only 1.0 μg of the biocytin-tRNA^{NNC} pool was added.

FIG. 8B shows binding of round 3 clones translated with increasing amounts of the biocytin-tRNA^{NNC} pool, indicating the level of selective pressure at each exogenous tRNA concentration for streptavidin binding, and revealing saturated sense suppression.

FIGS. 9A and 9B further demonstrate that iterative selection rounds result in the enrichment of sense suppression by orthogonal tRNAs. For these experiments, approximately 30 pmol of template with a fixed GUA codon was translated in the presence of 2.0 μg biocytin-acylated tRNAs containing the anticodon NAC, where the first position, N, is either U, C, A, or G.

FIG. 9A shows the sequences of 21 clones present after 4 rounds of selection on streptavidin-agarose. SEQ ID NO: 54 was found 16 times in the 21 clones sequenced, and each of SEQ ID NOS: 55 to 59 was found once.

FIG. 9B shows the biocytin incorporation in the GUA-containing template when different chemically acylated tRNAs were used. Incorporation was optimal when the anticodon was complementary to the GUA template codon. Values are expressed as the percent of (³⁵S)Met-labeled mRNA-peptide fusions bound to immobilized streptavidin.

FIG. 10A shows the results of sense and nonsense suppression comparisons performed in translation reactions containing approximately 30 pmol of each template with fixed codons as indicated. The minus sign (−) indicates percent binding (as in FIG. 8A) for translations performed without any exogenous tRNAs added. All other reactions contained 2.0 μg of the indicated biocytin-acylated tRNA. The plus sign (+) indicates translation reactions in the standard reaction; the single asterisk shows the binding results using a translation lysate that was passed over ethanolamine-SEPHAROSE gel to reduce endogenous tRNA concentrations prior to the suppression reaction (depleted); and the double asterisk (**) shows binding results obtained using the depleted translation lysate supplemented with 20 μg calf liver tRNA.

FIG. 10B shows the effect of the ethanolamine-SEPHAROSE gel treatment on fusion formation in the in-house translation extract (see Example 3). (+) and (−) as in FIG. 10A.

FIG. 10C shows the results of sense and nonsense suppression experiments carried out with the in-house prepared translation extract. (+) and (−) as in FIG. 10A. White bars show the suppression efficiency with the extract prior to modification. Light gray bars show suppression efficiency in the extract supplemented with calf liver tRNA. Black bars show the suppression efficiency in the extract that was depleted of tRNA using ethanolamine-SEPHAROSE gel treatment. The medium gray bars (right) show suppression efficiency in the extract that has been depleted of tRNA using ethanolamine-SEPHAROSE gel, then supplemented with calf liver tRNA.

FIGS. 11A to 11C show the strategy for synthesizing unnatural oligomers using mRNA display.

FIG. 11A shows the templates used. Template 41P (SEQ ID NO: 60) contains a single AUG codon. The 2G (SEQ ID NO: 61), 5G (SEQ ID NO: 62), and 10G (SEQ ID NO: 63) templates contain a single methionine codon followed by 2, 5, or 10 tandem GUA codons, respectively.

FIG. 11B shows the structure of the N-methyl-phenylalanine-acylated suppressor tRNA used.

FIG. 11C shows the expected structure of mRNA-peptide fusions constructed with 2G, 5G, and 10G (SEQ ID NOS: 61 to 64, respectively) templates in translation extracts containing no chemically acylated suppressor tRNA (left) or in extracts that have been supplemented with N-methyl-phenylalanine-acylated suppressor tRNA.

FIGS. 12A to 12C show the protease and nuclease sensitivity of (³⁵S)-Met-labeled products produced in the absence (−) or presence (+) of the chemically acylated N-methyl-phenylalanine suppressor tRNA^{UAC} as assayed by tricine SDS-PAGE. The 41P template (SEQ ID NO: 60) is included as a size and stability control.

FIG. 12A shows the protease and nuclease sensitivity of (35 S)-Met-labeled products made with the 2G template (SEQ ID NO: 61). The 41P template (SEQ ID NO: 60) is included as a size and stability control.

FIG. 12B shows the protease and nuclease sensitivity of [³⁵S]-Met-labeled products made with the 5G template(SEQ ID NO: 62). The 41P template(SEQ ID NO: 60) is included as a size and stability control.

FIG. 12C shows the protease and nuclease sensitivity of (³⁵S)-Met-labeled products made with the 10G template(SEQ ID NO: 63). The 41P template (SEQ ID NO: 60) is included as a size and stability control.

FIG. 13 shows the relative mobility and stability of (³⁵S)-Met-labeled products produced with the 41P, 2G and 5G templates (SEQ ID NOS: 60, 61 and 62, respectively) assayed using denaturing urea PAGE. The protease and nuclease sensitivity was examined for products produced in the absence (−) or presence (+) of the chemically acylated N-methyl-phenylalanine suppressor tRNA^{UAC}. This approach allows differences in the peptide portion of mRNA-peptide fusions to be analyzed as demonstrated previously (Roberts and Szostak, Proc. Natl. Acad. Sci. USA 94:12297-12302, 1997, which is incorporated herein by reference).

### DETAILED DESCRIPTION OF THE INVENTION

The present invention is based on a determination that diversity of peptide libraries can be increased by introducing or incorporating unnatural amino acids (i.e., amino acids other than the twenty naturally occurring amino acids) into one or more positions of peptides of the library. In particular, the incorporation of unnatural amino acid has been adapted to RNA display libraries, wherein the peptide containing the unnatural amino acid residue(s) is translationally linked to its encoding RNA to generate nucleic acid-peptide fusion molecules. Methods for producing mRNA display libraries containing the 20 naturally occurring amino acids are described, for example, in U.S. Pat. Nos. 6,281,344; 6,261,804; 6,258,558; 6,214,553; 6,207,446; each of which is incorporated herein by reference. The present invention extends the previously described methods by providing methods for introducing unnatural amino acid residues into peptides of a nucleic acid-peptide fusion molecules by post-translationally modifying one or more amino acid functional groups, and methods for translationally incorporating an unnatural amino acid residue into a peptide using one or more appropriate tRNA molecules such as orthogonal aminoacylated tRNA molecules, each charged with an unnatural amino acid residue.

Libraries of diverse nucleic acid-peptide fusion molecules are provided, wherein each molecule of the library comprises an encoding nucleic acid translationally linked to an encoded peptide, and wherein one or more peptides of the library contain at least one unnatural amino acid residue. As used herein, the term “encoding nucleic acid” refers to a nucleotide sequence that comprises two or more codons that can be translated into a peptide. Similarly, the term “encoded peptide” refers to an amino acid sequence that can be translated from an encoding nucleic acid. Generally, the encoding nucleic acid is an RNA molecule, which can be any RNA molecule that can be a template for translation of a peptide as disclosed herein, for example, an mRNA molecule transcribed from a DNA or RNA template, or a chemically synthesized RNA molecule.

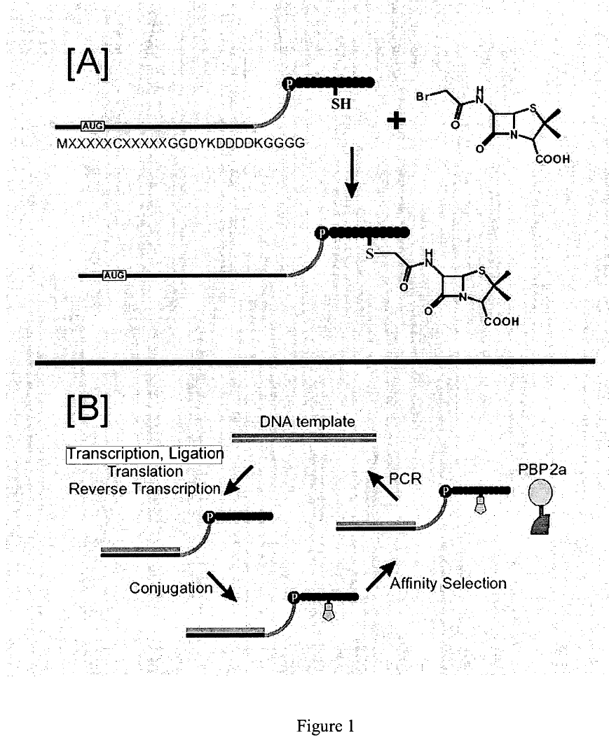

An advantage of the RNA display system is that an encoding nucleic acid is translationally linked to its encoded peptide (see, for example, U.S. Pat. No. 6,281,344). As used herein, the term “translationally linked” refers to the joining of an encoding nucleic acid and its encoded peptide during translation of the peptide due to the catalytic activity of a ribosomal peptidyl transferase. Generally, the encoding nucleic acid molecule is linked, either directly or indirectly via its 3′ terminus, to the encoded peptide, generally at the C-terminus of the peptide, during translation of the peptide (see FIG. 1; see, also, U.S. Pat. No. 6,281,344). A peptide and nucleic acid can be translationally linked using a peptide acceptor. As used herein, the term “peptide acceptor” means a molecule that can be added to the C-terminus of a growing (nascent) peptide chain due to the catalytic activity of the ribosomal peptidyl transferase function. A peptide acceptor, which is exemplified by puromycin, generally contains a nucleotide or nucleotide-like moiety such as adenosine or an adenosine analog (e.g., adenosine di-methylated at the N-6 amino position) linked to an amino acid or an analog or derivative thereof (e.g, O-methyl tyrosine; see, also, Ellman et al., Meth. Enzymol. 202:301, 1991, which is incorporated herein by reference), wherein the linkage is, for example, an ester, amide, or ketone linkage. Preferably, the linkage does not substantially perturb the pucker of the ring from the natural ribonucleotide conformation. A peptide acceptor also can contain a nucleophile, which can be, for example, an amino group, a hydroxyl group, or a sulfhydryl group. A peptide acceptor also can comprise a nucleotide mimetic, amino acid mimetic, or a mimetic of a combined nucleotide-amino acid structure.

In general, a peptide acceptor is positioned at the 3′ terminus of an encoding nucleic acid molecule. As such, the peptide acceptor molecule can be positioned immediately following the final codon of the peptide coding sequence, or can be separated from the final codon by a linker, for example, an intervening nucleotide sequence, which can be DNA or RNA. Preferably, the 3′ terminus of the peptide coding sequence, or the linker, when present, comprises a translation pause site. A peptide acceptor can be covalently bound to the peptide coding sequence of the nucleic acid, or can be linked non-covalently, for example, through hybridization using a second nucleotide sequence that selectively hybridizes at or near the 3′ end of the peptide coding sequence and that itself is bound to a peptide acceptor molecule, or that bridges and selectively hybridizes at or near the 3′ end of the peptide coding sequence and at or near a first end of a second nucleotide sequence, wherein the peptide acceptor is linked at or near the second end of the second nucleotide sequence.

A peptide acceptor is exemplified herein by puromycin, which resembles tyrosyl adenosine and acts to attach a growing peptide to its encoding mRNA (see U.S. Pat. No. 6,281,344). Puromycin is an antibiotic that acts as a chain terminator. As a mimetic of aminoacyl-tRNA, puromycin acts as a universal inhibitor of protein synthesis by binding the translation complex A site, accepting the growing peptide chain, and falling off the ribosome (at a Kd=10⁻⁴ M; Traut and Monro, J. Mol. Biol. 10:63, 1964; Smith et al., J. Mol. Biol. 13:617, 1965). Puromycin forms a stable amide bond to the growing peptide chain, thus allowing for more stable fusions than other peptide acceptors that form, for example, a less stable ester linkage. A peptidyl-puromycin molecule contains a stable amide linkage between the C-terminus of the nascent peptide (i.e., the peptide while it is still bound in the translation complex) and the O-methyl tyrosine portion of the puromycin. The O-methyl tyrosine is linked by a stable amide bond to the 3′-amino group of the modified adenosine portion of puromycin. As such, methods for translationally linking an encoding nucleic acid and encoded peptide are disclosed herein and include, for example, effecting the joining using a peptide acceptor such as puromycin, which can be linked at or near the 3′ terminus of the encoding nucleic acid such that it can enter the ribosome complex during translation and be incorporated at the C-terminus of the growing (nascent) peptide, thereby terminating translation and linking the encoding nucleic acid and encoded peptide.

Additional peptide acceptors useful for translationally linking an encoding nucleic acid and an encoded peptide include, for example, tRNA-like structures at the 3′ end of the mRNA, and other compounds that act in a manner similar to puromycin, for example, a compound that includes an amino acid residue linked to an adenine or an adenine-like compound (e.g., phenylalanyl-adenosine, tyrosyl adenosine, and alanyl adenosine), as well as amide-linked compounds such as phenylalanyl-3′-deoxy-3′-amino adenosine, alanyl-3′-deoxy-3′-amino adenosine, and tyrosyl-3′-deoxy-3′-amino adenosine. An example of a functional adenine-like compound is 7-deaza-adenosine (tubercidin) with a 3′ amino acid attached (see Krayevsky and Kukhanova, Prog. Nucl. Acids Res. 23:2-51, 1979, which is incorporated herein by reference). If desired, such peptide acceptors can contain a naturally occurring L-amino acid or contain an analog or derivative thereof, provided the peptide acceptor can translationally link the encoding nucleic acid and encoded peptide. A combined tRNA-like 3′ structure-puromycin conjugate also can be used as a peptide acceptor.

The encoding nucleic acid generally includes an RNA sequence, which includes a peptide coding sequence that can, but need not, be randomized, biased, or variegated in one or more positions. Such RNA coding sequences can be generated, for example, by in vitro transcription, from libraries of random, biased, or variegated oligodeoxyribonucleotides, wherein the oligonucleotide libraries are prepared using well known methods (see, for example, U.S. Pat. Nos. 5,622,699; 5,264,563; 5,223,409; 5,206,347; and 5,198,346; see, also, Scott and Smith, Science 249:386-390, 1992; Markland et al., Gene 109:13-19, 1991; O'Connell et al., Proc. Natl. Acad. Sci., USA 93:5883-5887, 1996; Tuerk and Gold, Science 249:505-510, 1990; Gold et al., Ann. Rev. Biochem. 64:763-797, 1995, each of which is incorporated herein by reference).

An unnatural amino acid can be any modified amino acid, including, for example, an amino acid analog, derivative or mimic. Thus, the term “unnatural amino acid” is used broadly herein to include any non-naturally occurring amino acid (i.e., an amino acid other than one of the 20 naturally-occurring amino acids). Unnatural amino acids are exemplified herein by biocytin, which is a biotin derivative of lysine; N-methyl-phenylalanine, an N-substituted amino acid; cysteine-thioether-6-amido penicillanic acid, which comprises a cysteine residue linked through its thiol group to a β-lactam antibiotic; α-hydroxy acids; α-sulthydryl acids; hydrazino amino acids; aminooxy amino acid; β-amino acids; amino acids modified at their side chains to contain a nucleoside, or nucleotide, or analog thereof, e.g., ATP, GTP, or analogs thereof; amino acid residues modified to contain a kinase inhibitor such as staurosporine, K252a, or derivatives thereof; and amino acid mimics such as peptoid monomers N-methyl glycine, N—(S)-phenylethyl glycine, or other N-substituted glycine. Additional examples of unnatural amino acids include any of the twenty naturally occurring amino acid that has been derivatized, for example, by modification of a functional group, particularly its side chain, e.g., a thiol group, an amino group, a carboxyl group, a guanidinium group, a hydroxyl group, or a phenolic group; which can be modified, for example, to a carboxylic acid, an acid halide, a carboxylic ester, a thioester, or a carbamate, and which can be further modified to contain a ligand or other desired moiety.

An advantage of preparing libraries containing, for example, peptides having an unnatural amino acid comprising a bound ligand (or other specific binding pair member) or any other molecule of interest (collectively “moiety”) is that, in the context of a peptide, the moiety can have desirable and unique properties. For example, the moiety can be ligand such as staurosporine, which binds to and inactivates several kinases (target molecule), including the calcium/calmodulin-dependent kinase, myosin light chain kinase, and protein kinases A, B and C (see, for example, Lazarovici et al., Adv. Exp. Med. Biol. 391:367, 1996; staurosporine and K252 compounds are available for example, from Alomone Labs; Jerusalem Israel) or a staurosporine derivative such as 7-hydroxystaurosporine (UCN-01) or N-benzoylstaurosporine (CGP 41251) (Gescher, Crit. Rev. Oncol. Hematol. 34:127, 2000). Upon preparing a library of nucleic acid-peptide fusion molecules, wherein peptides of the library contain unnatural amino acid residues comprising staurosporine, the library can be selected against one or a plurality of specific kinases in order to identify, for example, peptides that contain staurosporine in a context that renders the staurosporine specific only for one (or a few) particular kinase(s) or that increases the inhibitory effectiveness of the staurosporine (e.g., by decreasing the concentration required to inhibit a defined amount of kinase such as 50%). Similarly, the methods and compositions of the invention provide a means to identify agonist or antagonists of target molecules such as eukaryotic and/or prokaryotic enzymes, and proteins such as G proteins, which are involved in signal transduction. For example, a library containing peptides comprising an unnatural amino acid modified to contain GTP or a GTP analog can be screened against one or few different types of G proteins to identify peptides that present the GTP (or analog) in a context such that it exhibits a desirable agonist or antagonist activity and specificity.

The libraries of nucleic acid-peptide fusion molecules, preferably RNA-peptide fusion molecules, are characterized in that each fusion molecule of the library comprises an encoding nucleic acid linked to an encoded peptide, wherein peptides of the library contain at least one unnatural amino acid. The nucleic acid is translationally linked to the peptide using a peptide acceptor, which is present at or near the 3′ end of the encoding nucleic acid and can be added to the C-terminus of a growing polypeptide chain by the catalytic activity of a ribosomal peptidyl transferase. Since the libraries of the present invention are not limited to the 20 naturally occurring amino acids, they provide a much greater functional diversity and improved properties compared to libraries of peptides of similar size, and typically contain in excess of 10¹³ molecules, which is about 4-7 orders of magnitude greater than display libraries containing only naturally occurring peptides.

The peptide component of the nucleic acid-peptide fusion molecules can include any number of amino acid resides, from about 3 to 5 to more than 1000 (e.g., 5, 10, 15, 20, 25, 50, 100, 250, 500, 1000). As such, the term “peptide” is used in its broadest sense to refers to two or more amino acid residues, or derivatives or analogs thereof, linked by a bond. Generally, a peptide contains at least 3, 4, 5, 6, 7, 8, 9, 10, 15, 20, 25, 30, or more amino acid residues, and contain up to several hundred amino acid residues. As such, the term “polypeptide” can be used interchangeably with “peptide” , particularly where the peptide contains about 30 to 50 or more amino acid residues. In addition, the term “peptide” broadly includes proteins, which generally are naturally occurring polypeptides that can be post-translationally modified in vivo, for example, by glycosylation, by a proteolytic processing, and the like. For purposes of the present invention, the peptide component of a nucleic acid-peptide fusion molecule can be a peptide of any length, and can be a randomly generated peptide, or can be biased or variegated based, for example, on a portion of a naturally occurring protein such as a binding domain of a receptor (or ligand for a receptor), or a substrate binding domain of an enzyme (or a domain of a substrate comprising an amino acid that is modified by an enzyme), or an epitope of an antigen (or complementarity determining region of an antibody specific for an antigen).

The present invention provides methods of producing libraries of diverse nucleic acid-peptide fusion molecules, wherein peptides of the library contain one or more unnatural amino acid residues. In various embodiments, the unnatural amino acids are introduced into peptides of the library post-translationally by modifying one or more amino acid residues, or are incorporated into the peptides during translation using an appropriately charged orthogonal tRNA. Generally, translation of an encoding nucleic acid requires that the nucleic acid include a translation initiation sequence. As used herein, the term “translation initiation sequence” refers to any ribonucleotide sequence that enables initiation of protein synthesis. Translation initiation sequences are well known and include, for example, the Shine-Delgamo sequence in bacterial systems (Stormo and Gold, Nucl. Acids Res. 10:2971-2996, 1983, which is incorporated herein by reference) or a proper Kozak context around the initiator AUG codon in eukaryotic systems (Kozak, J. Biol. Chem. 266:29867-29870, 1991, which is incorporated herein by reference).

The term “orthogonal”, when used in reference to a tRNA molecule, means that the tRNA cannot be aminoacylated (“charged”) or edited by aminoacyl tRNA synthases present in a protein synthesis extract. An orthogonal tRNA is exemplified herein by THG73 (see Example 2). In addition to orthogonal tRNAs, other appropriate tRNAs can be used for purposes of the present invention, for example, when using a lysate that lacks tRNA synthetase activity such that the tRNA is not charged (aminoacylated) by an endogenous tRNA synthetase. Thus, for purposes of the present invention, an appropriate tRNA molecule such as an orthogonal tRNA is a tRNA that is (or can be) aminoacylated with an unnatural amino acid, which can be an analog or derivative of the amino acid that normally is bound to the tRNA or can be any other unnatural amino acid residue.

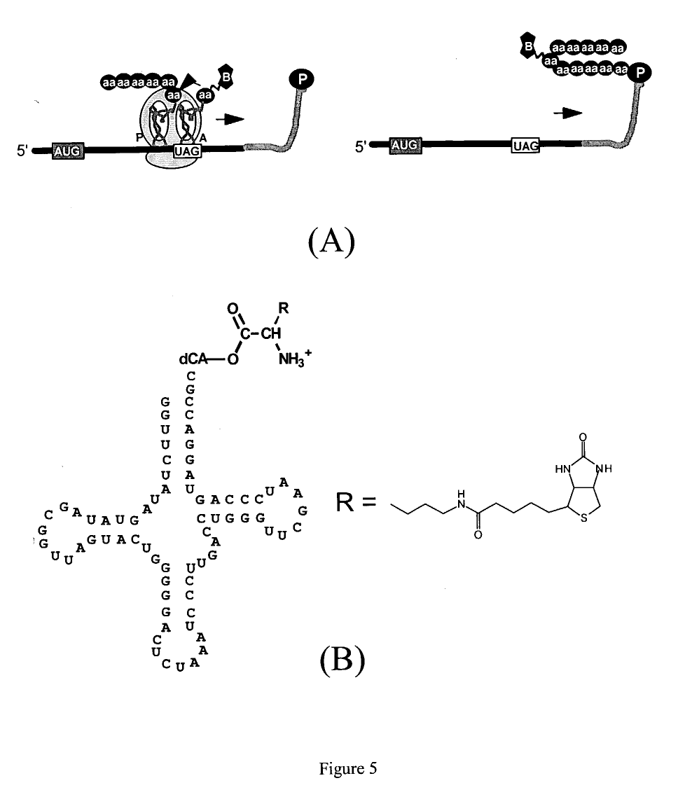

The term “stop codon” is used in the conventional and commonly understood sense to refers to a sequence of three nucleotides that normally signal termination of translation. Stop codons are well known, and include UAG, UGA, UAA. It will be recognized that, with respect to an embodiment of the present invention, the presence of stop codon in an encoding nucleic acid sequence may not, in fact, result in termination of translation, particularly when the translation is performed in the presence of a tRNA that suppresses termination of translation, including in the presence of a suppressor tRNA that is aminoacylated to contain an unnatural amino acid. Suppressor tRNA molecules include those known in the art as amber, ochre, and opal, and are exemplified herein by an amber suppressor tRNA, which is aminoacylated to contain an unnatural amino acid residue (see Example 2, and FIG. 5B). Thus, in its broadest sense, the term “orthogonal”, when used with respect to a tRNA, encompasses a suppressor tRNA (e.g., THG73) that is aminoacylated with an unnatural amino acid, since such an unnatural amino acid is not generally bound to a suppressor tRNA molecule in nature, and because an aminoacylated suppressor tRNA is be aminoacylated or edited by aminoacyl tRNA synthases, if any, present in a protein synthesis extract. A characteristic of an orthogonal tRNA aminoacylated with an unnatural amino acid is that the unnatural amino acid is not cleaved from the orthogonal tRNA due, for example, to the editing/proof-reading function of an aminoacyl tRNA synthetase that generally charges the tRNA.

According to one embodiment, a library of diverse nucleic acid-peptide fusion molecules, containing peptides with unnatural amino acids is produced by in vitro translating peptide coding sequences of a plurality of RNA molecules to generate nucleic acid-peptide fusion molecules, and contacting nucleic acid-peptide fusion molecules of the library with a peptide modifying agent under conditions suitable for post-translationally modifying at least one amino acid residue in one or more of the nucleic acid-peptide fusion molecules. The peptide modifying agent can be a chemical reagent that selectively modifies a functional group of one or more amino acid residue, or can be an enzyme that can selectively modify an amino acid residue. Preferably, the peptide modifying acts under conditions that do not substantially affect the nucleic acid-peptide fusion molecule (other than at the particular amino acid or residue to be modified), for example, by causing depurination or base hydrolysis of the nucleic acid component, particularly RNA, or by modifying or otherwise affecting reactive side chains of amino acids other than that to be modified.

As used herein, the term “peptide modifying agent” refers to a chemical, physical or biological agent that can alter an amino acid residue in a peptide. Such peptide modifying agents include any molecule that can react to form a covalent bond with a suitable functional group contained within the mRNA display library. A peptide modifying agent can modify a particular amino acid to generate the desired unnatural amino acid, or can modify the amino acid such that a second agent or reagent can be contacted with the modified amino acid to generate the desired unnatural amino acid. For example, a peptide modifying agent can be used to modify a thiol group, an amino group, a carboxyl group, a guanidinium group, a hydroxyl group, or a phenolic group of an amino acid, and can generate the desired unnatural amino acid, which comprises, for example, a carboxylic acid, an acid halide, a carboxylic ester, a thioester, a carbamate, a thiol group, an amino group, or a hydroxy group. Examples of peptide modifying agents that can react with one or more functional groups of an amino acid, particularly amino acid side chain functional groups, include N-hydroxysuccinimide (NHS), which reacts with the primary amine present in exposed lysine residues or an exposed N-terminus of the peptide, and maleimide, which reacts with the thio group present in cysteine; iodoacetyl and bromoacetyl groups, which modify cysteine residues; isothiocyanates, which modify primary amines; and hydrazides, which modify aldehyde and ketone groups.

Such modified amino acids can be used as unnatural amino acids, or can be further contacted, for example, with a moiety that has a reactive group than can react with the modified group on the amino acid, thus generating an unnatural amino acid comprising the moiety. Such a moiety can be any molecule as desired, including a biologically active molecule, an affinity tag, a detectable label such as a spectroscopic probe, a selectable marker, or other small organic molecule, peptide, protein, nucleic acid molecule, or the like. Affinity tags include, for example, a polyhistidine sequence, protein A (Nilsson et al., EMBO J. 4:1075, 1985; Nilsson et al., Methods Enzymol. 198:3, 1991), glutathione S transferase (Smith and Johnson, Gene 67:31, 1988), a Glu-Glu affinity tag (Grussenmeyer et al., Proc. Natl. Acad. Sci. USA 82:7952, 1985), substance P, FLAG peptide (Hopp et al., Biotechnology 6:1204, 1988), streptavidin binding peptide, biotin, or other antigenic epitope or binding domain (see, also, Ford et al., Protein Expression and Purification 2:95 (1991). Examples of spectroscopic probes, include, for example, Alexa Fluor, Marina Blue, Pacific Blue, Alexa Fluor 430, Fluorescein-EX, fluorescein isothiocyanate, Oregon Green 488, Oregon Green 514, Tetramethylrhodamine Red, Rhodamine Red-X, Texas Red, or other such probes (see, for example, Molecular Probes; Eugene, Oreg.).

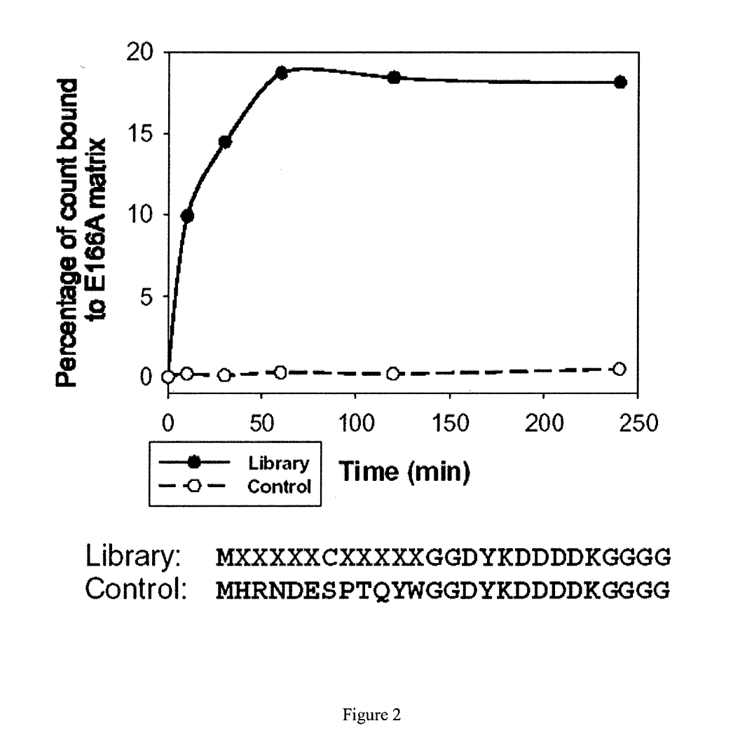

Aspects of the present invention are exemplified herein by post-translationally conjugating a cysteine residue with the β-lactam antibiotic, 6-bromoacetyl penicillanic acid (see FIG. 1, and Example 1). The engineered library was subjected to affinity selection against immobilized penicillin-binding protein 2a (PBP2a), which is a critical enzyme responsible for the drug resistance of methicillin-resistant Staphylococcus aureus (MRSA), and peptides having a greater affinity and, therefore, more likely to act as potent inhibitors were identified (see FIGS. 2 to 4). β-lactam drugs, antibiotics widely used to treat bacterial infections, are irreversible inhibitors of penicillin-binding proteins that are required by bacterial cell wall synthesis and essential for bacterial survival. Since the introduction of these drugs, numerous bacterial strains have gained resistance against them. β-lactamase, the primary reason responsible for bacterial resistance, can quickly destroy β-lactam antibiotics before they access their functional sites. Clinically, irreversible inhibitors of β-lactamases, including clauvlanic acid and sulbactam, are co-administrated with normal β-lactam drugs and have proven very efficient. However, the recent emergence of PBP2a, a unique penicillin-binding protein that remains functional at concentrations of β-lactam drugs high enough to inactivate other penicillin-binding proteins, imposes a serious health threat and necessitates the development of more powerful antibiotics. The present invention provides a means to identify agents that can be useful against otherwise drug resistant organisms, by allowing the selection of peptide/drug compositions (i.e., peptides containing unnatural amino acids comprising a drug moiety) having greater potency and/or selectivity.

Accordingly, the methods of the invention can further include a step of isolating nucleic acid-peptide fusion molecules from the library and, therefore, provides nucleic acid-fusion molecules selected by such a method. Methods for isolating nucleic acid-peptide fusion molecules from a library can utilize any procedure typical for isolating such molecules, provided the method does not damage the nucleic acid or peptide component of the fusion molecules, and does not disrupt the bond linking the components to each other. Convenient methods for isolating the fusion molecules include nucleic acid hybridization methods, for example, by including an oligonucleotide tag sequence as part of the nucleic acid component, and utilizing a complementary oligonucleotide, which can be linked to a solid support, to hybridize to and immobilize nucleic acid-peptide fusion molecules via the oligonucleotide tag. The tag, and complementary sequence, can be selected such that they selectively hybridize under conditions that are specific, but do not compromise the integrity of the fusion molecules. In one embodiment, an RNA-peptide fusion comprises an RNA containing a poly-A tail (e.g., an mRNA), in which case the RNA-peptide fusion can be isolated using oligo-dT cellulose (see Barrick et al., Methods 23:287-293, 2001, which is incorporated herein by reference).

Similarly, the nucleic acid component can contain a nucleotide sequence that encodes a peptide affinity tag, for example, the FLAG peptide sequence, which can be recognized by a specific antibody; the portion of protein A that binds to IgG; a sequence that can be biotinylated, which can bind to avidin or streptavidin; a polyhistidine sequence that can bind immobilized nickel ion; a calmodulin-binding peptide sequence; a chitin-binding domain; or the like (see, for example, Rigaut et al., Nat. Biotechnol. 17:1030-1032, 1999). An advantage of using a peptide tag to isolate fusion molecules of the library is that only those molecules that have undergone sufficient translation such that the peptide tag is synthesized are selected. If desired, the encoding nucleic acid can further encode, for example, a protease cleavage site such that a tag, or other such sequence, can be cleaved from the remainder of the peptide, particularly that portion of the peptide containing the unnatural amino acid residue(s).

In addition, the methods of the invention can include a step of contacting the nucleic acid-peptide fusion molecules, which comprise peptides containing unnatural amino acids, with a target molecule under conditions suitable for a specific interaction of the target molecule with a ligand specific for the target molecule; and such a method can further include a step of isolating nucleic acid-peptide fusion molecules that specifically interact with the target molecule. The term “target molecule” is used broadly herein to refer to any molecule that is being screened against nucleic acid-peptide fusion molecules of the library in order to identify peptides, which comprise at least unnatural amino acid, that can interact with the molecule. Such screening methods, which can be used to identify those fusion molecules having a desired property, can be any methods typically used, for example, to identify binding of a particular ligand to its receptor, or a particular substrate to an enzyme, or of an epitope to a specific antibody; or to identify agonist or antagonist activity with respect to the catalytic activity of an enzyme and its substrate, or to the binding activity of a receptor and its ligand. The present invention also provides nucleic acid-peptide fusion molecules isolated by such a method.

Based on the linked encoding nucleic acid sequence, a selected encoded peptide readily can be produced, in large amounts if desired, for example, by chemical synthesis of or reverse transcribing the encoding nucleic acid (RNA), PCR amplifying the reverse transcription product, and expressing the encoding nucleic acid (see below). Depending on whether the unnatural amino acid was introduced into the peptide post-translationally, or was incorporated into the peptide using an orthogonal aminoacylated tRNA, expression of an encoded peptide can be performed in vitro, for example, using a coupled in vitro transcription/translation system, or the expressible encoding nucleic acid can be introduced into a host cell for expression. In vitro translation generally is utilized where the unnatural amino acid is incorporated into the peptide using an orthogonal aminoacylated tRNA, whereas, when the unnatural amino acid is introduced post-translationally, an in vitro translation can be performed, or the peptide can be expressed in a host cell, then isolated from the cell, and the amino acid can be post-translationally modified in vitro. In addition, a selected peptide containing one or more unnatural amino acid residues can be prepared in a large or other desired amount using any method routinely used for the chemical synthesis of peptides, including, for example, using BOC chemistry or FMOC chemistry (see, for example, “Bioorganic chemistry: peptides and proteins” (Sidney M. Hecht editor, Oxford University Press, New York, N.Y., 1998), see Chapter 3, pp. 27-64, and Chapter 4, pp. 65-99; see, also, Hackeng et al, Proc. Natl. Acad. Sci. USA 96:10068-10073, 1999, each of which is incorporated herein by reference).

As described, in part, in U.S. Pat. No. 6,281,344, nucleic acid-peptide fusion molecules can be generated, first, by preparing a DNA template, then transcribing RNA to be used as the encoding nucleic acid, or by chemically synthesizing the RNA, including, as desired one or more randomized, biased, or variegated codons. Where a DNA template is used to produce the RNA, the template can be made using any standard technique, including methods of recombinant DNA technology, chemical synthesis, or both. Chemical synthesis can be used, for example, to produce a random cassette which is then inserted into the middle of a known protein coding sequence, thus providing a means to produce a high density of mutations around a specific site of interest in the protein (see, for example, chapter 8.2, Ausubel et al., Current Protocols in Molecular Biology, John Wiley & Sons and Greene Publishing Company, 1994). Random sequences may also be generated by the “shuffling” technique (Stemmer, Nature 370:389, 1994).

An alternative to total randomization of a DNA template sequence is partial randomization; a pool synthesized in this way is generally referred to as a “doped” pool (see, for example, Ekland et al., Nucleic Acids Research 23:3231, 1995). Partial randomization may be performed chemically by biasing the synthesis reactions such that each base addition reaction mixture contains an excess of one base and small amounts of each of the others; by careful control of the base concentrations, a desired mutation frequency may be achieved by this approach. Partially randomized pools also can be generated using error prone PCR techniques (see, for example, Beaudry and Joyce, Science 257:635, 1992; Bartel and Szostak, Science 261:1411, 1993).

To optimize a selection scheme of the invention, the sequences and structures at the 5′ and 3′ ends of an encoding nucleic acid can be altered. Preferably, this is carried out in two separate selections, each involving the insertion of random domains into the template proximal to the appropriate end, followed by selection. These selections can maximize the amount of fusion made (thus maximizing the complexity of a library) or can provide optimized translation sequences. Further, the method may be generally applicable, combined with mutagenic PCR, to the optimization of translation templates both in the coding and non-coding regions.

Where RNA is generated from a DNA template, in vitro transcription can be performed using, for example, T7 RNA polymerase, SP6 polymerase, T3 polymerase, and E. coli RNA polymerases. If desired, the synthesized RNA can be modified, in whole or in part, for example, by incorporating phosphorothioate bonds (using T7 polymerase), provided the modification does not substantially affect the ability of the RNA to be translated according to a method of the invention. An advantage of using such a modified RNA is that it is less susceptible to RNAse activity, thus allowing for a wider range of reactions to be used, for example, to post-translationally modify an amino acid to produce an unnatural amino acid.

In the next step of the aspect of the invention, puromycin (or any other appropriate peptide acceptor) is covalently bonded to the template sequence. This step can be accomplished, for example, by synthesizing an oligonucleotide (e.g., using an automated method) containing a 3′ puromycin, wherein the oligonucleotide containing the 3′ puromycin can be ligated to the nucleic acid template using a splint and T4 DNA ligase (see Liu et al., Meth. Enzymol. 317:268-293, 2000, which is incorporated herein by reference), or can be ligated to the template sequence photochemically using a puromycin oligonucleotide that further contains a 5′ psoralen (Kurz et al., Nucl. Acids Res. 28: E83, 2000, which is incorporated herein by reference). Alternatively, this step can be accomplished using T4 RNA ligase to attach the puromycin directly to the RNA sequence, or preferably the puromycin may be attached by way of a DNA “splint” (bridge) using T4 DNA ligase (see above) or any other enzyme which is capable of joining together two nucleotide sequences (see, also, Ausubel et al., supra, chapter 3, sections 14 and 15).

A tRNA synthetase also can be used to attach puromycin-like compounds to RNA. For example, phenylalanyl tRNA synthetase links phenylalanine to phenylalanyl-tRNA molecules containing a 3′ amino group, generating RNA molecules with puromycin-like 3′ ends (Fraser and Rich, Proc. Natl. Acad. Sci. USA 70:2671 (1973)). Other peptide acceptors which may be used include, without limitation, any compound which possesses an amino acid linked to an adenine or an adenine-like compound, such as the amino acid nucleotides, phenylalanyl-adenosine (A-Phe), tyrosyl adenosine (A-Tyr), and alanyl adenosine (A-Ala), as well as amide-linked structures, such as phenylalanyl 3′ deoxy 3′ amino adenosine, alanyl 3′ deoxy 3′ amino adenosine, and tyrosyl 3′ deoxy 3′ amino adenosine; in any of these compounds, any of the naturally occurring L-amino acids or their analogs may be utilized. A number of peptide acceptors are described, for example, by Krayevsky and Kukhanova (supra, 1979).

RNA-peptide fusions then are generated using an in vitro or in situ translation system. Virtually any in vitro or in situ translation system can be utilized, although eukaryotic systems are preferred, particularly the wheat germ and reticulocyte lysate systems. The rabbit reticulocyte translation extract provides the advantage of excellent stability of the template in this media and the efficiency of fusion formation. Translation systems constructed from isolated components or fractions of an extract also can be particularly useful for incorporating unnatural amino acid residues into an RNA display library. Such systems include partially reconstituted systems and totally reconstituted systems (Forster et al., Anal. Biochem. 297:60-70, 2001; Shimizu et al., Nat. Biotech. 19:751-755, 2001, each of which is incorporated herein by reference). However, any translation system that allows formation of an RNA-peptide fusion and does not significantly degrade the RNA portion of the fusion can be used. In addition, RNA degradation in any of these systems can be reduced or inhibited by including degradation-blocking antisense oligonucleotides in the translation reaction mixture; such oligonucleotides specifically hybridize to and cover sequences within the RNA portion of the molecule that trigger degradation (see, for example, Hanes and Pluckthun, Proc. Natl. Acad. Sci., USA 94:4937, 1997). Additional eukaryotic translation systems available for use in the present invention include, for example, lysates from yeast, ascites, tumor cells (Leibowitz et al., Meth. Enzymol. 194:536, 1991), and Xenopus oocytes. Useful in vitro translation systems from bacterial systems include, for example, those described in Zubay (Ann. Rev. Genet. 7:267, 1973); Chen and Zubay (Meth. Enzymol. 101:44, 1983); and Ellman (supra, 1991). In addition, translation reactions may be carried out in situ, for example, by injecting mRNA into Xenopus eggs using standard techniques.

Selection of a desired RNA-peptide fusion molecule, comprising a peptide containing an unnatural amino acid, can be accomplished by any means available to selectively partition or isolate a desired fusion from a population of candidate fusions. Examples of isolation techniques include, without limitation, selective binding, for example, to a binding partner which is directly or indirectly immobilized on a column, bead, membrane, or other solid support, and immunoprecipitation using an antibody specific for the protein moiety of the fusion. The first of these techniques makes use of an immobilized selection motif that can consist of any type of molecule to which binding is possible. These motifs include proteins, carbohydrates, RNA, DNA, transition state analogs, small molecules, etc. Selection also can be based upon the use of substrate molecules attached to an affinity label, for example, substrate-biotin, which reacts with a candidate molecule, or upon any other type of interaction with a fusion molecule. In addition, proteins can be selected based upon their catalytic activity. Accordingly, desired molecules are selected based upon their ability to link a target molecule to themselves, and the functional molecules are then isolated based upon the presence of that target.

Selection of a desired RNA-peptide fusion (or its DNA copy) can be facilitated by enrichment for that fusion in a pool of candidate molecules. To carry out such an optional enrichment, a population of candidate RNA-peptide fusions can be contacted with a binding partner, which is specific for either the RNA portion or the protein portion of the fusion, under conditions that substantially separate the binding partner-fusion complex from unbound members in the sample. This step can be repeated, and can include at least two sequential enrichment steps, one in which the fusions are selected using a binding partner specific for the RNA or linker portion and another in which the fusions are selected using a binding partner specific for the protein portion.

If enrichment steps targeting the same portion of the fusion molecule (e.g., the peptide component) are repeated, different binding partners can be utilized. In one particular example, a population of molecules is enriched for desired fusions by first using a binding partner specific for the RNA portion of the fusion, then, in two sequential steps, using two different binding partners, both of which are specific for the protein portion of the fusion. Again, these complexes can be separated from sample components by any standard separation technique including, without limitation, column affinity chromatography, centrifugation, or immunoprecipitation.

Elution of an RNA-peptide fusion from an enrichment (or selection) complex can be accomplished by a number of approaches, including, for example, a denaturing or non-specific chemical elution step to isolate a desired RNA-peptide fusion. Such a step can facilitate the release of complex components from each other or from an associated solid support in a relatively non-specific manner by breaking non-covalent bonds between the components and/or between the components and the solid support. An exemplary denaturing or non-specific chemical elution reagent is 4% HOAc/HO. Other exemplary denaturing or non-specific chemical elution reagents include guanidine, urea, high salt, detergent, brief exposure to elevated pH, or any other means by which non-covalent adducts can generally be removed. Alternatively, a specific chemical elution approach can be used, wherein a chemical is exploited that causes the specific release of a fusion molecule. For example, if a linker arm of a desired fusion protein contains one or more disulfide bonds, bound fusion aptamers can be eluted by the addition of a reducing agent such as DTT, resulting in the reduction of the disulfide bond and release of the bound target.

Elution also can be accomplished by specifically disrupting affinity complexes; such techniques selectively release complex components by the addition of an excess of one member of the complex. For example, in an ATP-binding selection, elution is performed by the addition of excess ATP to the incubation mixture. An enzymatic elution also can be used, wherein a bound molecule itself or an exogenously added protease (or other appropriate hydrolytic enzyme) cleaves and releases either the target or the enzyme. For example, a protease target site can be included in either of the complex components, and the bound molecules eluted by addition of the protease. Alternately, in a catalytic selection, elution can be used as a selection step for isolating molecules capable of releasing (for example, cleaving) themselves from a solid support.

A DNA copy of a selected RNA fusion sequence can be prepared by reverse transcribing that RNA sequence using any standard technique, for example, using SUPERSCRIPT reverse transcriptase. This step can be carried out prior to or following a selection or enrichment step, or can be carried out prior to the isolation of the fusion from the in vitro or in situ translation mixture. The DNA template then can be amplified, either as a partial or full-length double stranded sequence. Preferably, full length DNA templates are generated, using appropriate oligonucleotides and amplification, typically PCR amplification. The full length DNA temples can then be subjected to further rounds of in vitro translation and affinity selection, i.e., the resulting RNA-peptide fusion molecules can be subjected to repeated rounds of selection and amplification because the protein sequence information can be recovered by reverse transcription and amplification, for example, by PCR amplification or any other amplification technique, including RNA-based amplification techniques such as self-sustained sequence replication (3SR; Gingeras et al., Ann. Biol. Clin. 48:498-501, 1990). The amplified nucleic acid can then be transcribed, modified, and in vitro or in situ translated to generate mRNA-peptide fusions for the next round of selection. The ability to carry out multiple rounds of selection and amplification enables the enrichment and isolation of very rare molecules, e.g., one desired molecule out of a pool of 10¹⁵ members, which, in turn, allows the isolation of new or improved proteins that can specifically recognize virtually any target or catalyze (or inhibit) any desired chemical reaction.

In one embodiment, the present invention provides a method for producing an mRNA display library incorporating unnatural amino acids by in vitro translating protein coding sequences in the presence of a series of aminoacylated tRNA molecules at least one of which includes an unnatural amino acid residue, and incubating the translation reaction under high salt conditions to produce a population of RNA-peptide fusion molecule. Each protein coding sequence of each RNA molecule is linked to a peptide acceptor at the 3′-end of the peptide coding sequence, the peptide acceptor being a molecule that can be added to the C-terminus of a growing protein chain by the catalytic activity of a ribosomal peptidyl transferase. The aminoacylated tRNA molecule that is charged with an unnatural amino acid residue can recognize a codon that typically is recognized by a tRNA charged with a naturally-occurring amino acid (i.e. a codon that encodes an amino acid), or can recognize at least one stop codon. Using such methods of the invention, libraries of molecules having a diversity of at least about 109 different molecules, generally at least about 10¹¹ to 10¹² molecules, usually at least about 10¹³ molecules, and particularly more than 10¹³ molecules can be generated.

Many of the steps for performing this aspect of the invention using orthogonal aminoacylated tRNA molecules are identical to those discussed above, except that in the previous embodiment, one or more RNA-peptide fusion molecules that include an unnatural amino acid are produced after in vitro translation, i.e., post-translationally. In the present embodiment, the peptides containing unnatural amino acids are produced during in vitro translation by including at least one aminoacylated tRNA that is charged with an unnatural amino acid. According to the present method, however, the in vitro translation reaction generally is performed under standard conditions, followed by the addition of high salt (generally a concentration of a monovalent cation of at least about 200 mM and/or a concentration of a divalent or higher valence cation of at least about 25 mM), combined with incubation at low temperature (Liu et al., supra, 2000). The high salt incubation ensures that a significant fraction of the nascent peptide becomes attached to the template.

In one aspect of this embodiment, the orthogonal aminoacylated tRNA, which is charged with an unnatural amino acid residue, recognizes a stop codon, and one or more encoding nucleic acid sequence contains one or more stop codons. Accordingly, the method can be performed, for example, by providing a population of RNA molecules (e.g., mRNA molecules), each of which comprises a translation initiation sequence and a start codon operably linked to a protein coding sequence, and each of which is linked to a peptide acceptor at the 3′-end of the peptide coding sequence, which includes or can include one or a series of stop codons, and wherein the peptide acceptor is a molecule that can be added to the C-terminus of a growing protein chain by the catalytic activity of a ribosomal peptidyl transferase; contacting the RNA molecules, under conditions suitable for translation, orthogonal aminoacylated tRNA molecules charged with unnatural amino acid residues; and in vitro translating the peptide coding sequences of the RNA molecules in the presence of the orthogonal aminoacylated tRNA molecules, under high salt conditions, to produce a population of RNA-peptide fusion molecules comprising peptides containing one or more unnatural amino acid residues.

The genetic code is imposed, in part, by the presence of aminoacyl tRNA synthetases. These enzymes match tRNAs with their corresponding amino acids, defining the identity of individual codons. The ability to insert unnatural residues using a suppressor tRNA demonstrates that an appropriate chemically aminoacylated tRNA can be used as a stoichiometric reagent in the creation of the libraries of the invention. Thus, in the practice of the present invention, a set of chemically charged appropriate tRNAs such as orthogonal aminoacylated tRNAs can be generated and used produce libraries of nucleic acid-peptide fusion molecule comprising peptides having any number of unnatural amino acid residues, including peptides that are composed entirely of unnatural amino acids. Alternatively, a modified tRNA synthase can be used to enzymatically recharge an appropriate tRNA as exemplified by the in vivo incorporation of fluorphenylalanine into proteins in bacteria via a yeast aminoacyl tRNA synthase (Furter, Prot. Sci. 7:419-426, 1998, which is incorporated herein by reference).

The incorporation of an unnatural amino acid residue using a chemically charged orthogonal tRNA is exemplified using the unnatural amino acid residue, biocytin (see Example 2). Biocytin, a biotin derivative of lysine, represents an exemplary choice for incorporation into an unnatural peptide library because it can be incorporated into proteins (Gallivan et al., Chem. Biol. 4:739, 1997, which is incorporated herein by reference), and because the biotin moiety can be used to select sequences that have incorporated the biocytin, for example, using a streptavidin-, monoavidin-, or avidin-containing solid support. As exemplified, the biocytin charged aminoacylated tRNA was based on an amber suppressor tRNA, which recognizes a UAG stop codon. However, other unnatural residues such as N-methyl glycine and N—(S)-phenylethyl glycine similarly can be used. An orthogonal aminoacylated tRNA also can be one that recognizes a codon for a specific naturally occurring amino acid residue, but is charged with an unnatural amino acid, which, can, but need not, be based on the naturally occurring residue. Thus, according to the methods of the invention, numerous different unnatural amino acid residues can be incorporated into peptides, including, for example, 1, 2, 3, 4, 5, 10, 15, 20, 25, 30, 50, or more unnatural amino acids.

The methods of the invention utilizing appropriate aminoacylated tRNA molecules such as orthogonal aminoacylated tRNA molecules charged with unnatural amino acids involves reprogramming the genetic code, and requires that the translation system allows insertion of unnatural residues and does not compete with the unnatural amino acids for insertion of natural amino acids. The reticulocyte lysate translation system can be particularly useful for purposes of this aspect of the invention (Jackson et al., Meth. Enzymol. 96:50, 1983), although other systems such as an E. coli (see, for example, Chen and Zubay, supra, 1983; Ellman et al., supra, 1991) or wheat germ extract (Madin et al., Proc. Natl. Acad. Sci., USA 97:559, 2000; Bain et al., Nature 356:537, 1992, each of which is incorporated herein by reference) based translation system also can be used. Partially or totally reconstituted translation systems also can be useful for the insertion of unnatural amino acids (Forster et al., supra, 2001; Shimizu et al., supra, 2001).

Chemically acylated orthogonal tRNAs are used to insert the unnatural residues. Synthesis of such tRNAs has been worked out in detail (Saks et al., J. Biol. Chem. 271:23169-23175, 1996, which is incorporated herein by reference; Ellman et al., supra, 1991). In these protocols, L-amino acids are N-acylated with the NVOC photolabile protecting group, coupled to a dCpA dinucleotide, and ligated to an appropriate tRNA. The amino acids must have an L-configuration at the a-carbon or be achiral (e.g., glycine or N-substituted glycines; Noren et al., Science 244:182-187, 1989; Bain et al., supra, 1992). The amine nitrogen can be replaced by an OH (α-hydroxy acids; Dougherty, Curr. Opin. Chem. Biol. 4:645, 2000, which is incorporated herein by reference) or by a hydrazine (Killian et al., J. Amer. Chem. Soc. 120:3032, 1998, which is incorporated herein by reference).

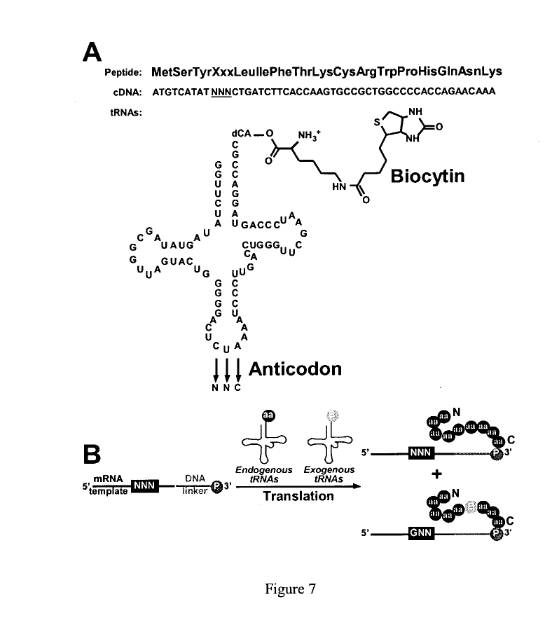

Insertion at positions other than an amber stop codon (code=UAG) requires the design of tRNAs that bind to the other triplet codons. Although tRNA synthetases can recognize such tRNAs and remove the chemically acylated residues (editing) or charge the tRNAs with natural amino acids, orthogonal tRNAs can be created that are not recognized by any synthetase using the universal identity rules for eukaryotic tRNAs (Giege et al., Nucl. Acids Res. 26:5017, 1998, which is incorporated herein by reference). As such, THG73 (SEQ ID NO: 25) was be used as an initial template for the design of these tRNAs (Saks et al., supra, 1996; see Example 3; FIG. 7A). THG73 appears to be orthogonal to the eukaryotic amino acyl tRNA synthase (aaRS) when the anticodon contains UAG. Thus, THG73 variants containing 16 natural anticodons were constructed and orthogonal variants were identified (Example 3) The results of Example 3 demonstrate that THG73 variants containing the 61 natural anticodons can be generated and tRNA variants that remain orthogonal can be identified. Modifications of other identity positions (e.g., the acceptor stem) also can be made if insertion of anticodons provides editing or acylation with the translation extract.

The UAG stop codon has often been referred to as a “blank” in the code because it can be suppressed by an appropriate tRNA (Noren et al., supra, 1989). However, the activity of specific aminoacyl tRNA synthases (aaRS) can be eliminated, creating specific “blanks” in the code. Various high affinity inhibitors of the synthetases can be generated to create synthetic blanks in the translation extracts. All of the aaRS act by using ATP to create an activated form of an amino acid, a mixed carbon-phosphorus anhydride termed an aminoacyl adenylate. Aminoacyl sulfamides are stable structural mimics of aminoacyl adenylates and generally have submicromolar Kᵢ values for their corresponding synthetase (see U.S. Pat. No. 5,824,657; Tao and Schimmel, Exp. Opin. Invest. Drugs 9:1767-1775, 2000, each of which is incorporated herein by reference). For example, sulfamides of Phe, Leu, Ile, Met, and Val all have Kᵢ values under 20 nM with the corresponding HeLa aaRS. Addition of micromolar concentrations of synthetically constructed sulfamides can specifically block the action of one or more specific synthetases in a predefined manner.

For this aspect of the invention, aminoacylated tRNAs present in the in vitro translation reaction, typically a cell lysate, can also compete with the chemically acylated tRNAs for insertion, even when the synthetases are inhibited. These activities can be removed in multiple ways. First, nuclease treatment can be performed to remove functional tRNAs. This treatment, using micrococcal nuclease, is performed on the presently used lysates to remove the endogenous globin templates (Jackson and Hunt, Meth. Enzymol. 96:50, 1983). Second, the tRNAs in the lysate can be deacylated. For example, addition of Cu (II) ions greatly facilitates the hydrolysis of aminoacyl tRNAs (Schofield and Zamecnik, Biochim. Biophys. Acta 155:410, 1968). Following such a reaction, the copper can be sequestered by adding EGTA or EDTA to the extract. Finally, the reticulocyte lysate translation system contains considerable RNase H activity, which cleaves the RNA portion of an RNA/DNA hybrid. As such, endogenous tRNAs can be destroyed using biotinylated DNA oligonucleotides (5′-TGG-biotin) complementary to the acceptor end universal in all tRNAs (5′-CCA). Alternatively, specific tRNAs can be targeted using DNA oligonucleotides complementary to the anticodon loop. The biotinylated oligonucleotides can then be removed with streptavidin agarose after treatment.

In certain embodiments, the unnatural amino acid can include a biologically active agent, an affinity tag, and/or a spectroscopic probe, as described above. For example, the biologically active agent can be staurosporine, a staurosporine derivative such as UCN-01, CGP41251, or a K252a compound. Staurosporine, a protein kinase C inhibitor, is also known to inhibit other protein kinases when used at higher concentrations. Accordingly, libraries of RNA-peptide fusion molecules can be constructed using the methods of the present invention, that include an unnatural amino acid residue with a staurosporine moiety. Such staurosporine-containing RNA-peptide fusion molecules include molecules that can be useful for treating cancer and other cellular proliferative disorders in which protein kinase C is known to have a role in the etiology.

Libraries of staurosporine-containing PNA-peptide fusion molecules can be used to produce more powerful kinase inhibitor or to change the kinase specificity of staurosporine, by subjecting the staurosporine-containing RNA-peptide fusion molecules to affinity selection using a protein kinase linked to a solid support. For example, to produce staurosporine-containing peptides that have a higher affinity to protein kinase C than staurosporine, multiple rounds of affinity selection can be performed using higher stringency binding conditions in subsequent selection rounds, for example by including shorter incubation times for a binding step. As another example, to produce staurosporine-containing peptides with altered specificity, the affinity selection procedure can be carried out using a kinase such as the Ca²⁺/calmodulin-dependent kinase, cAMP dependent kinase (PKA), Ca²⁺/phospholipid-dependent kinase (PKC), or cGMP-dependent kinase.

A biologically active molecule present on the unnatural amino acid can be a peptide known to bind important protein domains. For example, peptides can bind to two well known SH3 domain sequences from Grb2 and Crk. SH3 domains play an important role in signal transduction by acting as modular interaction domains, binding proline-rich peptides in other proteins. Previously, Lim and coworkers have studied interactions with the peptide YEVPP₂PVP₋₁PRRR (SEQ ID NO: 47), substituted at the P₂ and P₋₁ proline residues (numbered based on their relationship to the central valine; Nguyen et al., Science 282:2088, 1998, which is incorporated herein by reference). Substitution for proline with N-methyl glycine at P₂ and P₋₁ produces little change in the binding to both the Grb2 and Crk N terminal SH3 domain. Interestingly, N—(S)-phenylethyl glycine produced an enhancement of −2.0 kcal/mol at P₂ in Crk and −2.0 kcal at P₋₁ in Grb2 (Nguyen et al., supra, 1998). An RNA-peptide fusion library randomized at the P₂ and P₋₁ positions can be generated and selected for molecules that bind to either Grb2 or Crk. Grb2 and Crk can be expressed and biotinylated specifically in vivo by including a short biotinylation sequence in the N-terminus or C-terminus (Beckett et al., Prot. Sci. 8:921, 1999, which is incorporated herein by reference) to allow linking of Grb2 and Crk to a solid support. Chemically acylated tRNAs, as discussed above, can be used for the selection with an appropriate codon library. For example, using the UAG suppressor, the library would be randomized (C/U) (C/A) G codons (to capture the CCG=proline or UAG=suppressor) at the P₂ and P₋₁ locations.

In other embodiments, unnatural amino acid residues are included that provide functionalization after synthesis or allow cyclization of the libraries (i.e., allow formation of cyclic peptides). For example, inclusion of ketones in the unnatural amino acids provides a means to specifically derivatize peptides (Mahal et al., Science 276:1125, 1997, which is incorporated herein by reference). Also residues that contain carbon-carbon double bonds can be inserted. For example, insertion of two allyl glycine residues allows cyclization of peptides via ring-closing metathesis (Miller et al., J. Amer. Chem. Soc. 118:9609, 1997; Schafmeister et al., J. Amer. Chem. Soc. 122:5891, 2000, each of which is incorporated herein by reference). Furthermore, residues that contain carbon nucleophiles in place of the normal primary amine can be included in the unnatural amino acids. Many carbon nucleophiles pose difficulty as they are not water stable (e.g., Gringard reagents). However, if carbon nucleophiles are included in the unnatural amino acids, and act in the catalytic center of the ribosome, all carbon-scaffold products would be constructed in an encoded fashion to achieve a completely nonpeptidic ribosomal product.

In one example of this aspect of the invention, the series of appropriate aminoacylated tRNAs, e.g., orthogonal aminoacylated tRNAs, can include the entire population of aminoacylated tRNAs in a translation reaction, and each orthogonal aminoacylated tRNA can be charged with an unnatural amino acid, thereby producing a peptide that is entirely or predominantly made up of unnatural amino acids. For this aspect, endogenous tRNAs present in an in vitro translation reactions, typically cell lysates, can be removed by any of a series of methods. For example, the in vitro translation reaction can be treated with micrococcal nuclease before adding the aminoacylated tRNA charged with an unnatural amino acid. This treatment has already been performed on in vitro translation reactions to remove endogenous globin templates (Jackson et al., supra, 1983).

In another aspect, a set of one or more chemically aminoacylated tRNAs is generated, which are added to a translation extract that is dependent on exogenous tRNAs for function. A standard nuclease treated reticulocyte system prior to supplementing with tRNAs is one example of a suitable translation extract. An exemplary tRNA is the amber suppressor tRNA THG73 (a modified Tetrahymena thermophila Gln tRNA). This tRNA can be used to insert an unnatural residue, for example a biocytin moiety (see FIG. 5B) by nonsense UAG suppression, as this construct has high efficiency in eukaryotic translation systems. Translation of templates in this system generates libraries with chemical structures based on the selected codon pairs. For example, a chemically aminoacylated tRNA containing a UAC anticodon (e.g., a valine tRNA) can be acylated with N-ethyl glycine. Chemical acylation can be performed by a variety of methods well known to those skilled in the art. Using this tRNA in a translation reaction results in incorporation of N-methyl phenylalanine at every residue where the codon GUA was encountered in the template (see Example 4).

This aspect of the invention provides libraries that differ markedly from natural peptides and proteins. In addition, to increase the diversity of the mRNA libraries, the monomers in the libraries can include other molecules, particularly amino acid mimics, in place of amino acids. For example, the monomers can be N-substituted amino acids, α-hydroxy acids, and the like, as well as combinations thereof. Indeed, it will be understood that any monomer compatible with a translation reaction can be incorporated into invention libraries, thus creating an enormous diversity of encoded libraries.

The following examples are intended to illustrate but not limit the invention.

### EXAMPLE 1

### PBP2A INHIBITORS ISOLATED FROM AN mRNA DISPLAY LIBRARY CONTAINING PEPTIDES HAVING A PENICILLIN SIDE CHAIN

The Example illustrates a general strategy to introduce unnatural side chains into mRNA display libraries via post-translational modification.

As disclosed herein, by iteratively selecting an mRNA-display library containing a β-lactam moiety against penicillin-binding protein 2a (PBP2a), inhibitors of PBP2a having improved affinity were obtained. This method provides a convenient way to generate very large libraries of molecules, including drug based molecules and to enhance the efficacy of known ligands, and facilitates the discovery of ligands with functionalities beyond those provided by the 20 naturally occurring residues.

Combinatorial peptide libraries are rich reservoirs for sieving novel ligands of many therapeutically interesting targets, including agonists or antagonists of receptors, epitopes of antibodies, and inhibitors of enzymes. Techniques such as phage, ribosome, and mRNA-display libraries can generate peptides that are physically associated with their own genes, making it easy to identify molecules with desired properties after iterative cycles. However, the chemical diversity of these libraries is restricted to the 20 naturally occurring amino acids. By contrast, synthetic peptide libraries can contain unnatural amino acids in desirable positions, but they are not amplifiable and the identification of active molecules requires tedious deconvolution processes or sophisticated encoding strategies.

In vitro translation of mRNA molecules modified with puromycin at the 3′-end results in formation of a covalent bond to the encoded peptides, and can provide up to 10¹³ independent peptide sequences in a selectable format (see, for example, Takahashi et al., Trends Biochem. Sci. 28:159-165, 2003; Roberts, Curr. Opin. Chem. Biol. 3:268-273, 1999, each of which is incorporated herein by reference). As disclosed herein, methods have been developed to introduce unnatural amino acid residues into the peptide component of mRNA display libraries posttranslationally, or translationally using an amber stop codon (see Example 2) or sense suppression (see Examples 3 and 4). This example illustrates that an unnatural β-lactam side chain can be incorporated into an mRNA display library via posttranslational modification. The library then was subjected to affinity selection against immobilized PBP2a, which is a critical enzyme responsible for the drug resistance of methicillin-resistant Staphylococcus aureus (MRSA; see Hiramatsu et al., Trends Microbiol. 9:486-493, 2001, which is incorporated herein by reference), to sieve for more potent inhibitors.

β-lactam drugs, which include antibiotics widely used to treat bacterial infections, are irreversible inhibitors of penicillin-binding proteins that are required by bacterial cell wall synthesis and essential for bacterial survival. Since the introduction of these drugs, numerous bacterial strains have gained resistance against them. β-lactamase, the primary reason responsible for bacterial resistance, can quickly destroy β-lactam antibiotics before they access their functional sites. Clinically, irreversible inhibitors of β-lactamases, including clauvlanic acid and sulbactam, are co-administrated with normal β-lactam drugs and has proven very efficient. However, the recent emergence of PBP2a, a unique penicillin-binding protein that is still functional at concentrations of various β-lactam drugs high enough to inactivate other penicillin-binding proteins, imposes a more serious health threat and necessitates the development of more powerful antibiotics.

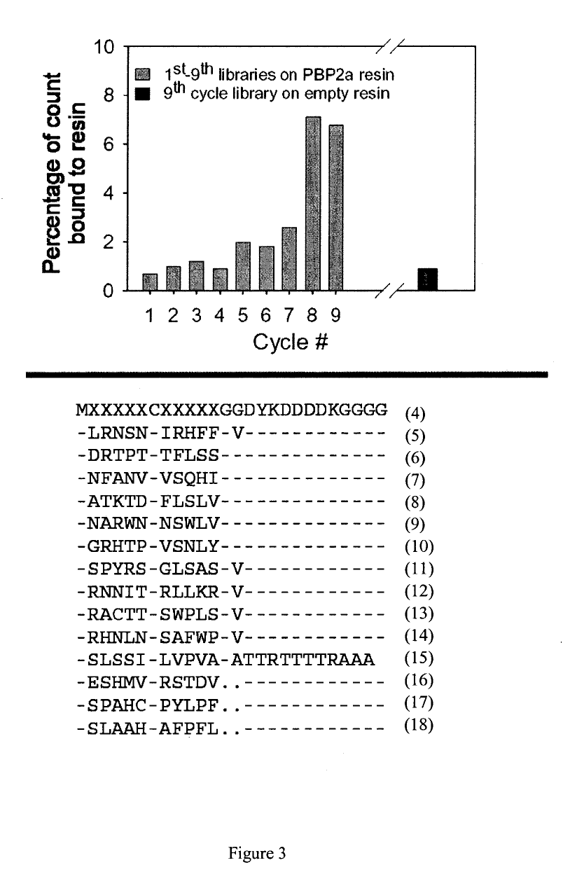

As disclosed herein, an mRNA peptide library was constructed in which a constant cysteine was flanked on each side by 5 random residues (Li and Roberts, Chem. Biol. 10:233-239, 2002, which is incorporated herein by reference; see FIG. 3). The constant cysteine residue was modified with an unnatural β-lactam side chain through an orthogonal coupling reaction between the thiol group and 6-bromoacetylpencillanic acid (FIG. 1A). As such, a “hybrid” library was generated that represented a huge collection of diverse β-lactam compounds with various peptides appended at the 6-position of the β-lactam ring. Numerous modifications at this position to enhance the pharmacokinetics and other properties of β-lactam drugs have been described (Kidwai et al., Curr. Med. Chem. 6:195-215, 1999, which is incorporated herein by reference). Several inhibitors of PBP2a were identified from this mRNA display peptide library containing the unnatural β-lactam side chain (FIG. 3; SEQ ID NOS: 5-18).

Method's

Preparation of Sodium 6-bromoacetylpenicillanate

432 mg of 6-aminopenicillanic acid (Sigma, 6-APA, 2 mmol) and 500 mg sodium bicarbonate (Sigma, 4.5 mmol) were mixed in 4 ml water and 2 ml acetone with stirring for 10 min at 0° C. 404 mg of bromoacetyl bromide (Aldrich, 2 mmol, 175 μl) was dissolved in 2 ml of acetone and added to the stirring solution. The reaction flask was wrapped with aluminum foil and kept on ice for an additional 10 min. The solution was removed from the ice bath and allowed to come to room temperature. The reaction was stirred for 1 hr, and 5 ml of water was added to dissolve any residual white salt in the flask. The reaction was extracted twice with 4 ml ether, then covered with 5 ml ethyl acetate. The reaction mixture was acidified with 40% phosphoric acid with stirring at 0° C. The ethyl acetate layer was removed, extracted (3×) with 5 ml distilled water and dried over anhydrous magnesium sulfate. The dried reaction mixture was combined with 300 μl n-butanol containing sodium 2-ethylhexanoate and stirred for 30 min. The precipitate was collected by filtration, washed with several portions of ethyl acetate, and air dried (395 mg). The ESI-MS gives two equal peaks at 335.0 and 337.0; the expected mass peaks (M−H)⁻ are 334.97 and 336.97.

Synthetic DNA Library Template

sd7: 5′-ACTATTTACAACCACCATGNNSNNSNNSNNSNNSTGCNNSNNS NNSNNSNNSGGCGGCGACTACAAGGACGACGATGACAAGGGCGGCGGCG GC-3′ (SEQ ID NO: 1; ATG start codon in bold) was purified by preparative polyacrylamide gel electrophoresis and amplified by polymerase chain reaction (PCR) with 2 primers,

| | | |
|---|---|---|
| sd2: | 5′-GGATTCTAATACGACTCACTATAGGGACAATTACTATTTACAACCACC | (SEQ ID NO:2) |
| | ATG-3′, and | |
| sd3: | 5′-GCCGCCGCCGCCCTTGTCATCGTCGTCCTTGTAGTC-3′. | (SEQ ID NO:3) |

The resulting double-stranded DNA template was used to prepare the mRNA display fusion library as previously described (see U.S. Pat. No. 6,281,344). Fusion were purified using oligo-dT cellulose (New England Biolabs), then used as templates for reverse transcription at conditions recommended by the manufacturer (SUPERSCRIPT II RNase H⁻ reverse transcriptase; Life Technologies). The product was purified by phenol extraction and ethanol precipitation and dissolved in 300 μl reaction buffer (100 mM borate, pH=8.3), then 8 μl tris(2-carboxyethyl) phosphin (TCEP, 20 mM, pH=8.0) was added and the reaction incubated at room temperature for 30 min. 1.1 mg sodium bromoacetyl penicillanate (10 mM final solution) was dissolved in the reaction solution, and the mixture was shaken for 1 hr in the dark. The reaction was quenched by adding 30 μl 2M DTT, and the reaction was allowed to continue for an extra 30 min. The fusion conjugate was ethanol precipitated and dissolved in water prior to selection.

Selection Against Immobilized PBP2a

The truncated PBP2a gene (Roychoudhury et al., J. Biol. Chem. 269:112067-12073, 1994, which is incorporated herein by reference) was inserted into PTXB3 plasmid (New England Biolabs) between Nco I and Sap I sites, then transformed into E. coli BL21(DE3) strain. Bacteria were grown in LB medium at 37° C. until A₆₀₀ reached 0.6, then were induced with 1 mM IPTG. After growing for an additional 3 hr at 37° C., the bacteria were harvested for purification of PBP2a protein using chitin beads, as recommended by manufacturer (New England Biolabs). The PBP2a protein was cleaved from beads with 50 mM DTT in column buffer (20 mM HEPES, pH=8.0; 500 mM NaCl; 1 mM EDTA) and dialyzed against phosphate buffer (50 mM, pH=8.0) overnight.

Pure PBP2a protein was reacted with biotin-SS-NHS (Pierce Chemical) and dialyzed against phosphate buffer (50 mM pH=8.0). The resulting biotinylated PBP2a was incubated with streptavidin agarose gel and shaken for 30 min. After draining extra PBP2a solution and washing with phosphate buffer, the fusion conjugate was mixed with immobilized PBP2a agarose in 1 ml of incubation buffer (50 mM phosphate, pH=8.0; 100 mM NaCl; 2 mM biotin) for variable times. The agarose was washed with 6×500 μl washing buffer (50 mM phosphate, pH=8.0; 100 mM NaCl; 0.1% Triton-X100), then incubated with 500 μl cleavage buffer (50 mM phosphate, pH=8.0; 100 mM NaCl; 100 mM DTT) for 1 hr. The supernatant was collected, desalted, concentrated using a MICROCON Y-30 microconcentrator (Millipore), and used for PCR amplification directly with sd2 (SEQ ID NO: 2) and sd3 (SEQ ID NO: 3) as primers.

Relative IC₅₀ Assay of Peptide Penicillanate Conjugates

The selected peptides were synthesized in an ABI peptide synthesizer with Fmoc chemistry, then deprotected with TFA and purified by HPLC. The lyophilized peptides were reacted with sodium bromoacetyl penicillanate in borate buffer (100 mM, pH=8.3). The conjugates of peptide with β-lactam were purified again by HPLC and lyophilized. Various concentration of conjugates were prepared by dissolving in reaction buffer (50 mM phosphate, pH=8.0; 100 mM NaCl) and incubating with 2 Vg pure PBP2a respectively at 37° C. for 15 min. 20 μg biotinylated ampicillin was added immediately and incubated at 37° C. for 10 min. The reaction was stopped by heating the reaction mixture at 94° C. for 5 min. The samples were loaded into 12% SDS-PAGE for separation, then were transferred to a nitrocellulose membrane by blotting with sodium bicarbonate buffer (20% methanol; 3 mM Na₂CO₃; 10 mM NaHCO₃) at 400 mA for 3 hr. The membrane was blocked with blocking solution (20 mM Tris, pH=8.0; 150 mM NaCl; 0.05% TWEEN-20 detergent; 5% milk) for 1 hr, then incubated with 10 ml TBS buffer (20 mM Tris, pH=8.0; 150 mM NaCl) containing 1 μg/ml streptavidin-HRP protein (Pierce Chemical) at room temperature for 1 hr. The membrane was washed 6 times with 50 ml TBS buffer, at 5 min intervals, and covered with FEMTO-WESTERN blotting reagent (Pierce Chemical). The membrane was then examined under a digital camera at dark to collect chemiluminescent emission for 10-15 min.

Results

Based on the efficiency of mRNA peptide fusion formation, the library contained approximately 1.5×10¹³ unique peptides, thus representing saturation coverage of peptide libraries with 10 randomized positions (20¹⁰=10¹³). As such, the full sequence space of 10-mer peptides could be searched during the selection. The constant cysteine residue in freshly-made libraries was in a reduced state and ready for conjugation with 6-bromoacetyl penicillanic acid.

The mRNA display peptides modified by 6-bromoacetylpencillanic acid were indistinguishable from those not containing the β-lactam side chain in standard gels because the molecular weight difference between them is too small to be resolved. In order to test the coupling efficiency of the mRNA peptide library with 6-bromoacetyl penicillanic acid, a mutant β-lactamase was made that replaced the Glu166 with an Ala residue. The enzymatic reaction of the wild type β-lactamase involves a two-step mechanism. First, the β-lactam molecule forms an acyl-intermediate with β-lactamase through Ser70. Then, the water molecule inside the active pocket is activated by Glu166, and sequentially attacks the covalent intermediate to release the degraded product. The substitution of the key residue Glu166 by alanine dramatically reduces the hydrolysis rate of the enzyme, resulting in the accumulation of acyl-enzyme intermediate (Escobar et al., Biochemistry 30:10783-10787, 1991). Thus, when treated with mutant β-lactamase (E166A), the peptide fusions containing the β-lactam can recruit the mutant enzyme E166A, a 30 kD protein, and shows much slower electrophoretic mobility than those not yet modified. Approximately 20% of the mRNA-peptide fusions formed a conjugate with 6-bromoacetyl penicillanic acid, indicating that the low concentration of mRNA-peptide fusion in the coupling mixture (usually less than 100 nM) rendered the reaction incomplete. Accordingly, the starting library contained approximately 3×10¹² different peptides bearing the β-lactam drug. Equally important, the bromoacetyl drug was chemically orthogonal with the functional groups on the template (hydroxyls, phosphates, ring nitrogens, exocyclic amines) and the non-cysteine amino acids (histidine, arginine, asparagine, glutamic and aspartic acid, serine, threonine, glutamine, tyrosine, lysine and tryptophan), as well as the N-terminal amine in the peptide. Finally, these experiments demonstrate that the penicillin side chain can undergo a covalent attachment interaction with the active site of β-lactamase when covalently attached to a great variety of peptide chains.

A control template was constructed that contained all other functional groups provided by the remaining 19 naturally occurring amino acids, to demonstrate that 6-bromoacetyl penicillanic acid reacts exclusively with cysteine. Both the control and library fusions were treated with 6-bromoacetyl penicillanic acid and incubated with immobilized mutant β-lactamase (E166A) matrix. The resulting control peptide fusions were unable to adsorb onto matrix coated with immobilized E166A mutant, while approximately 20% of library peptide fusions remained bound after extensive washing (FIG. 2). This result indicates that the other 19 naturally occurring amino acids (i.e., other than Cys), as well as the four ribonucleotides in the RNA template linked with each peptide, are inert to bromoacetyl modification.

Successive cycles of in vitro selection and amplification were performed by applying the β-lactam tagged 10-mer peptide fusion library on immobilized PBP2a matrix. Because the β-lactam moiety can react with PBP2a slowly to form a covalent adduct, the incubation time of the ‘hybrid’ library on the PBP2a matrix was gradually shortened from 1 hr in the first cycle to 10 min in the ninth cycle in order to enrich peptide β-lactam conjugates that are kinetically advantageous.

After the completion of nine cycles of selection and amplification, the binding of libraries arising from each cycle on PBP2a matrix was examined by incubating the libraries with matrix for 30 min. The fraction of libraries bound to the immobilized PBP2a beads increased from 0.4% of the first cycle library to 7% of the 9^{th} cycle library, while that of the ninth cycle library against blank solid support remained only 0.6% (FIG. 3). Fifteen individuals from the 9^{th} cycle library were cloned and sequenced. All of the sequences contained a cysteine residue at the fixed position, and one of sequences appeared twice (FIG. 3; SEQ ID NOS: 5-18). The sequence analysis revealed various mutations and deletions in the constant region of the templates. The fact that only two cysteines appeared in randomized positions of these 15 clones indicated that affinity improvement due to the enrichment of cysteines did not dominate the pool. No obvious consensus was found between these clones, indicating that many distinct peptide can enhance the binding and/or reactivity of the β-lactam core.

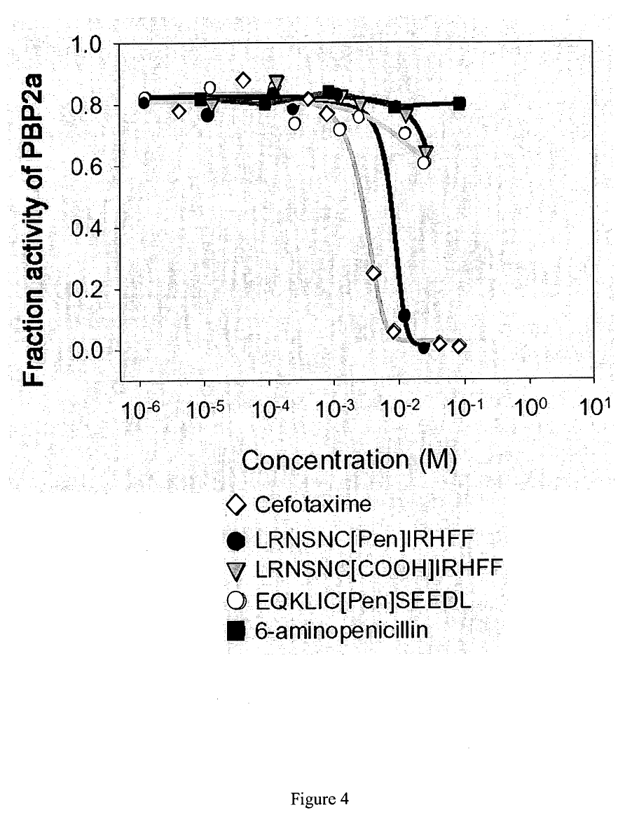

One peptide from the selection (SEQ ID NO: 5) was synthesized milligram quantities to test their inhibition against PBP2a. This peptide was derivatized with 5-bromopenicillanic acid to give the product (LRNSNC(pen)IRHFF; residues 2-12 of SEQ ID NO: 5). As a control, the peptide also was derivatized with bromoacetic acid to give a carboxylate in place of the penam drug, (LRNSNC(COOH)IRHFF; residues 2-12 of SEQ ID NO: 5). Reacting the cysteine with bromoacetic acid also provide the benefit that the modification precludes intermolecular disulfide bond formation (see Le and Roberts, supra, 2003). The resulting peptide conjugate and other control compounds were analyzed in a competition assay (Dargis and Malouin, Antimicrob. Agents Chemother. 38:973-980, 1994, which is incorporated herein by reference). The relative IC₅₀ of this peptide conjugate (LRNSNC(pen)IRHFF; residues 2-12 of SEQ ID NO: 5) was about 7 mM, making it more potent as a PBP2a inhibitor than the unmodified peptide (IC₅₀>35 mM) or 6-aminopenicillanic acid (6-APA; IC₅₀>500 mM; FIG. 4). A control peptide conjugate (EQKLIC(pen)SEEDL; SEQ ID NO: 19) that did not appear in the final enriched library showed no such improvement (IC₅₀>35 mM). Thus, the selected peptide conjugate was about 100-fold improved as compared to 5-APA, and was comparable to cefotaxime (IC₅₀=2.9 mM), a cephalosporin compound (FIG. 4). These results demonstrate that substitution at the 6-position of the β-lactam ring with various peptides derived from the selection can enhance their affinity against PBP2a cooperatively. Optimization of the linkage between the β-lactam and peptide moieties can further be examined to identify enhanced potency of the selected peptide against PBP2a.

The modest IC₅₀ (7 mM) observed does not necessarily imply that binding of our substrates is correspondingly weak. First, the absolute value of the IC₅₀ depends on the conditions of the assay, with longer conjugate-PBP2a incubation times producing correspondingly smaller IC₅₀ values. Additionally, the rate constant for covalent attachment of penams to PBP2a (k₂) is quite slow, ranging from k₂=0.22 sec⁻¹ for benzyl penicillin to k₂=0.0083 sec⁻¹ for methicillin (Lu et al., Biochemistry 38:6537-6546, 1999). For comparison, the rate constant for formation of the acyl intermediate between penams and β-lactamase is ˜2000 sec⁻¹, which is about 10⁴-fold to 10⁶-fold faster than PBP2a (Christensen et al., Biochem. J. 266:853-861, 1990). Covalent attachment of the selected peptides depends on the ratio of k₋₁/k₂, as this value reflects how bound drug will partition between dissociation and product formation. Values for k₁, the formation rate constant are typically in excess of 10⁸ M⁻¹ sec⁻¹ for penams interacting with β-lactamase. If k₋₁˜k₂ in order to give significant product formation, the predicted value of K_{d} would be nanomolar or below. If it is conservatively estimated that k₁ is 10⁶ M⁻¹ sec⁻¹, typical for macromolecular association reactions, predicted values of K_{d} are in the micromolar range or below. Overall, the biochemical analysis indicates that the appended peptide facilitates the desired function of the drug by at least 100 fold. This observation is consistent with the selected peptides adding approximately 3 kcal to the stabilization (k₁/k₋₁), the reactivity (k₂), or a combination thereof to the parent penam drug.

The methods disclosed herein provide a means to extend the chemical diversity possible in mRNA display libraries through the creation of functional drug-peptide conjugates, as exemplified by the demonstration that a penicillin side chain can be appended to an mRNA display library in a chemically orthogonal fashion with reasonable synthetic efficiency to generate compounds having increased affinity for a penicillin binding protein. After 9 rounds selection, all of the cloned sequences were in frame and contained a cysteine residue at the fixed position. These results indicate that the peptide-drug conjugate was formed and selected for interaction with the PBP2a, targeting the peptide library to the active site of the protein. Chemical synthesis of the peptide-drug conjugate confirmed that this compound was active against PBP2a, whereas neither the drug nor the peptide, alone, had appreciable activity at concentrations that can be examined experimentally.

A compelling aspect of these results is their generality. There are numerous examples where drugs target specific sites in proteins (see, for example, Kuntz et al., Proc. Natl. Acad. Sci., USA 96:9997-10002, 1999; Christopoulos, Nat. Rev. Drug Discovery 1:198-210, 2002). The ability to generate peptide-drug conjugates enables the use of small molecules to direct display libraries to a particular face or site in a protein target of interest. Additionally, the covalent derivatization strategy disclosed herein enables library construction using compounds that may not be conveniently incorporated into a peptide using in vitro nonsense suppression. For example compounds that strongly inhibit translation such as kinase inhibitors or GTP analogs, or side chains that are too large to fit in the exit tunnel of the ribosome (e.g., a heme group), may not be efficiently inserted by translation using nonsense and/or sense suppression.

The disclosed method provides a means to introduce one or more unnatural amino acid residues into peptides of an mRNA-display peptide library. The chemistry that can be utilized in this posttranslational modification process is not limited to the reaction between cysteine and compounds containing bromoacetyl functionality. Reagents commonly used for protein labeling, such as N-hydroxysuccinimide (NHS), a reagent specifically reactive to primary amines at N-terminus or exposed lysine residues, and maleimide, specifically reactive to thio groups in cysteines, can also allow the construction of new molecular tools based on known pharmacophores. These libraries provide a convenient way to enhance the efficacy of therapeutically useful small molecules. For example, peptides containing an ATP analog that mimics the transition state of the phosphorylation reaction can be sieved for highly specific inhibitors against various protein kinases. The present method also provides a tool to aid physical organic dissection of protein interfaces, particularly where molecular interaction display context dependence. In particular, the disclosed methods can facilitate the discovery of novel ligands with functionalities beyond those provided by the 20 naturally occurring residues.

### EXAMPLE 2

### TRANSLATIONAL INCORPORATION OF UNNATURAL AMINO ACID RESIDUES INTO AN RNA DISPLAY LIBRARY

This example illustrates that in vitro selection experiments can be performed on RNA display libraries containing the unnatural amino acid biocytin (FIG. 5A).

Methods for incorporating unnatural amino acids into peptides using an orthogonal suppressor tRNA recognizing a stop codon have been described (Noren et al., supra, 1989; see, also, Ellman et al., supra, 1991). This unnatural strategy allows proteins to be constructed that contain a novel residue at a desired location, enabling insertion of affinity tags (Gallivan et al., supra, 1997), spectroscopic probes (Cornish et al., Proc. Natl. Acad. Sci., USA 91:2910-2914, 1994), and analogs for detailed mechanistic analysis (Arslan et al., J. Amer. Chem. Soc. 119:10877-10997, 1997). This suppression strategy has also been extended to eukaryotic systems both in vitro (Kargino et al., Nucleic Acids Res. 25:3912-3916, 1997, which is incorporated herein by reference; see, also, Bain et al., supra, 1992) and in vivo (Saks et al., supra, 1996; Wang et al., Science 292:498-500, 2001).

In mRNA display, translation extracts are used to generate combinatorial libraries of peptides, which are covalently linked to their encoding mRNA via a 3′-puromycin. These libraries are strictly monovalent and provide for the synthesis of greater than 10¹³ independent peptide sequences in a selectable format. The present method extends the previously described methods to provide a method for translationally introducing unnatural amino acid residues into a growing peptide.

Methods

Synthesis of Biocytin-tRNA Suppressor

Synthesis of NVOC-Biocytin Cyanomethyl Ester

One hundred mg Biocytin (0.26 mmol, Molecular Probes) and 56 mg sodium carbonate (0.54 mmol) were dissolved in a mixture of water (15 ml) and THF (10 ml). A solution of 6-nitroveratryloxycarbonyl chloride (NVOC-Cl; 74 mg, 0.26 mmol; Sigma Chemical Co.; St. Louis Mo.) in 10 ml THF was added slowly. After 3 hr, solvents were removed in vacuo, then 3 ml of dry DMF, 3 ml of chloroacetonitrile, and 800 μl triethylamine was added into remaining residues. After overnight stirring, solvents were removed in vacuo and the remaining solid was purified by flash chromatography (silica gel, 10% MeOH in CH₂Cl₂).

Preparation of Biocytin-dpCpA

Ten mg of tetrabutylammonium salt of dinucleotide (dpCpA; 8.3 μmol) and 16 mg NVOC-biocytin cyanomethyl ester (25 μmol) were mixed at room temperature in dry DMF under argon. Twenty μl ammonium acetate (25 mM, pH 4.5) was added to quench the reaction after 1 hr stirring. The crude product was purified by reversed phase semi-preparative HPLC using a gradient from 25 mM NH₄OAc (pH 4.5) to CH3CN. The appropriate fractions were combined and lyophilized. The resulting solid was redissolved in 10 mM acetic acid/CH3CN and lyophilized to give 3 mg biocytin-dpCpA as a pale yellow solid. The product was confirmed by mass spectrum as it gives a peak (M+H)⁺ at MW=1230.4.

In vitro Transcription of tRNA

THG73 tRNA was synthesized in vitro from Fok I linearized plasmid harboring THG73 tRNA gene using T7 MEGAshortscrip™ kit (Ambion). The product was purified by polyacrylamide gel electrophoresis and dissolved in water.

Ligation of Biocytin to THG73 tRNA

A mixture of THG73 tRNA.(25 μg in 10 μl water) and HEPES (20 μl, 10 mM, pH 7.5) was heated at 94° C. for 3 min, then slowly cooled to 37° C. Eight μl biocytin-dpCpA (3 mM in DMSO), 32 μl 2.5× reaction buffer (25 μl 400 mM pH 7.5 HEPES; 10 μl 100 mM DTT; 25 μl 200 mM MgCl₂; 3.75 μl 10 mM ATP; 10 μl 5 mg/ml BSA; 26.25 μl water; 1 μl Rnasin (Promega)), 5 μl water, and 5 μl T4 RNA ligase (New England Biolabs) was added. After 1 hr incubation at 37° C., the reaction mixture was extracted once with an equal volume of phenol (saturated with 300 mM sodium acetate, pH 5.0):CHCl₃:isoamyl alcohol (25:24:1), then precipitated with 3 volume of cold ethanol at −20° C. The precipitate was washed with cold 70% (v/v) ethanol, dried under vacuum, and resuspended in 5 μl 1 mM sodium acetate, pH 5.0. The amount of biocytin-tRNA was quantified by measuring A260 and the concentration was adjusted to 1 μg/μl with 1 mM sodium acetate (pH 5.0). Prior to the suppression reaction, the biocytin tRNAs solution was deprotected by xenon lamp equipped with a 315 nm cut-off filter for 5 min.

Construction of mRNA-peptide Fusions

Construction of Fusion Template

Synthetic DNA templates including

| | | |
|---|---|---|
| Pep 1: | 5′-ACTATTTACAACCACCATGGGCCGCCAGGAGATCCACTGGGCCAACG | (SEQ ID NO:20) |
| | ACCTGTGCAAGCCCTTCTGGGTGTACACCTCC-3′, | |
| Pep2: | 5′-ACTATTTACAACCACCATGGGCTAGCTTGACTACAAGGACGAGGACA | (SEQ ID NO:21) |
| | AGCGCCAGGAGATCCACTTGGCCAACGACCTGTGCAAGCCCTTCTGGGTGTA | |
| | CACCTCC-3′, and | |
| Lib1: | 5-ACTATTTACAACCACCATGGGCCGCCAGGAGATCCACNNSGCCAACG | (SEQ ID NO:22) |
| | ACCTGTGCAAGCCCTTCTGGGTGTACACCTCC-3′ | |

Lib1: 5-ACTATTTACAACCACCATGGGCCGCCAGGAGATCCACNNSGCCAACG ACCTGTGCAAGCCCTTCTGGGTGTACACCTCC-3′ (SEQ ID NO: 22), were purified by preparative polyacrylamide gel electrophoresis.

Polymerase chain reaction (PCR) of these templates was performed using two synthetic primers:

| | | |
|---|---|---|
| sd2: | 5′-GGATTCTAATACGACTCACTATAGGGACAATTACTATTTACAACCACC | (SEQ ID NO:2) |
| | ATG-3′ and | |
| sd26: | 5′-GCCGCCGCCGCCGGAGGTGTACACCCAGAAG-3′, | (SEQ ID NO:23) |

generates double stranded DNA. mRNA was produced by T7 runoff transcription of these templates in the presence of RNAsecure™ (Ambion) followed by size exclusion column purification (NAP25 column; Amersham Pharmacia Biotech).

A flexible DNA linker containing puromycin, F30P (5′-dA21{C9}3dACdCP (C9=triethylene glycol phosphate; P=CPG-puromycin, Glen Research), was synthesized using standard chemistry. The oligonucleotide was chemically phosphorylated using PHOSPHORYLATION REAGENT II reagent (Glen Research) and purified using an OPC cartridge. Ligation of pF30P to transcribed mRNA was done by mixing mRNA, pF30P, a splint, which has the sequence 5′-TTTTTTTTTTTTGCCGCCGCCGCC-3′ (SEQ ID NO: 24) in a ratio of 1:0.5:1.2 with 2 Units of T4 DNA ligase (New England Biolabs) per picomole of template mRNA. After ligation, the fusion template was gel purified, electroeluted and desalted by ethanol precipitation.

Translation and Fusion Formation

The fusion template was translated in reticulocyte lysate (Novagen) using standard conditions (800 nM template) with the addition of 35S-methionine as the labeling reagent. In the case of templates containing a UAG stop codon, 2 μg of deprotected biocytin-tRNA suppressor also was added. On completion of translation, fusion formation was stimulated by addition of MgCl₂ and KCl to 50 mM and 0.6 M, respectively, and incubated at −20° C. overnight. The resulting 35S-labeled mRNA peptide fusions were directly loaded to 15% tricine SDS-PAGE for separation. Following separation, the gel was dried and exposed to phosphor screen (Molecular Dynamics) for several hours. The phosphor screen was scanned to produce an image (FIG. 2B).

Enrichment of UAG Stop Codon by Selection Against Streptavidin-Agarose Matrix Template-Base (dT) Purification

To isolate fusion molecules, the lysate was diluted in binding buffer (1M NaCl, 20 mM Tris, pH 8.0, 1 mM DTT, 10 mM EDTA, 0.2% TRITON X-100 detergent) and incubated with dT-cellulose at 4° C. for 1 hr. Bound fusions were washed with washing buffer (0.3 M NaCl, 20 mM Tris pH 8.0) and eluted using ddH₂O.

Reverse Transcription and Selective Step

Following dT purification, fusion molecules were concentrated and used for reverse transcription with SUPERSCRIPT II RNase H⁻ reverse transcriptase (Life Technologies) following standard conditions recommended by the manufacturer. The reaction mixture (50 μl) was added directly into 1 ml phosphate buffer (50 mM, pH 7) and streptavidin-agarose matrix (Pierce). After a 1 hr incubation at 4° C., the matrix was washed with washing buffer (50 mM phosphate pH 8.0, 100 mM NaCl, 0.1% SDS) 500 μl×6 times. The matrix then was used for PCR amplification with sd2 (SEQ ID NO: 2) and sd26 (SEQ ID NO: 23). The PCR product was cloned with TOPO Clone kit (Invitrogen) for sequencing.

Results

Nucleic acid-peptide fusion molecules were synthesized to contain the unnatural amino acid residue, biocytin, which is a biotin derivative of lysine. Biocytin was selected for this study because it has been inserted into proteins (Gallivan et al., supra, 1997), and because the biotin moiety can be used to select peptides that have incorporated this amino acid derivative. mRNA display libraries were constructed in the rabbit reticulocyte translation extract, which demonstrates excellent stability of the template in this media and the efficiency of fusion formation.

The amber suppressor tRNA THG73 (SEQ ID NO: 25; a modified Tetrahymena thermophila Gln tRNA) was used to insert the unnatural amino acid residue by nonsense UAG suppression (FIG. 5B) as this construct has high efficiency in eukaryotic translation systems (Saks et al., supra, 1996). Two templates were constructed to test insertion of the unnatural residue—the first template (Pep1) was a control encoding all 20 amino acids, but no stop codon, while the second template (Pep2) encoded a similar peptide and also contained a single UAG stop codon at the third position. For Pep2, fusion formation occurred only when the suppressor tRNA was added, consistent with incorporation of biocytin into the Pep2 mRNA-peptide fusion.

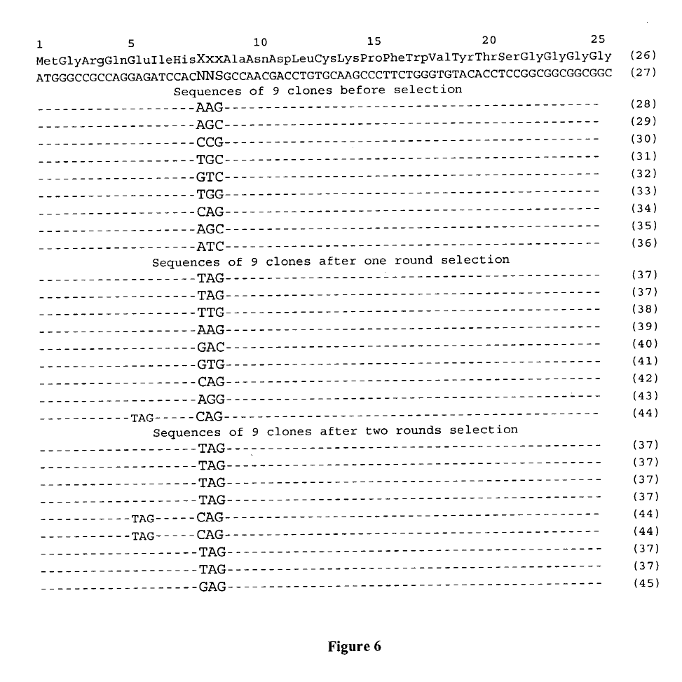

In order to demonstrate that this method could be used to select peptides from libraries based on the function of the unnatural residue, the TTG codon that encoded the Trp residue in position 8 of the template Pep1(not shown, but see SEQ ID NO: 26) was replaced with an NNS saturation cassette containing 32 possible codons encoding all 20 possible amino acids and the UAG stop (Lib1; see SEQ ID NO: 27). Two rounds of in vitro selection were performed using streptavidin agarose as an affinity matrix. Sequencing after one round of selection indicated that UAG stop codons were being enriched at both the randomized position and elsewhere in the open reading frame via point mutations (FIG. 6). After a second round of selection against streptavidin agarose, nine clones were sequenced from the library. Eight out of nine (88%) contained a UAG stop codon at the randomized position or elsewhere, including two that contain a GAG-to-UAG transversion at position 5 (FIG. 6).

These results demonstrate the successful combination of in vitro selection and nonsense suppression, thus allowing selectable peptide libraries to be constructed containing any unnatural amino acid that is compatible with the translation apparatus. These libraries can facilitate the discovery of ligands having functionalities beyond those available using peptides containing only the 20 naturally occurring amino acid residues. For example, peptides containing N-substituted amino acids are protease-resistant (Miller et al., Drug Devel. Res. 35:20-32, 1995) and can show enhanced affinity for natural protein interaction modules such as SH3 domains (Nguyen et al., supra, 1998). This method also can aid physical organic dissection of protein interfaces, particularly where molecular interactions display context dependence. Additionally, this approach provides a convenient way to construct new molecular tools based on known pharmacophores, in that therapeutically useful small molecules can be presented adjacent to the chemical diversity present in a 10¹³-member peptide or protein library.

### EXAMPLE 3

### TRANSLATIONAL INCORPORATION OF UNNATURAL AMINO ACID RESIDUES USING SENSE SUPPRESSION OF GUA, GCG, and GCU CODONS

This Example demonstrates that tRNA molecules specific for sense codons can be used to incorporate unnatural amino acids into peptides.

The results disclosed in Example 2 demonstrate that the unnatural residue biocytin can be incorporated into mRNA display libraries using the THG73 amber suppressor tRNA chemically-acylated with the unnatural amino acid (see, also, Saks et al., J. Biol. Chem. 271:23169-23175, 1996), and that mRNA display can be used to select mRNA templates capable of efficiently incorporating an unnatural amino acid, specifically mRNA templates containing a UAG codon complementary to the anticodon in the THG73 suppressor tRNA. This Example extends those results by demonstrating that sense codons also can be targeted for tRNA mediated incorporation of unnatural amino acid residues (“sense suppression”).

An mRNA display-based strategy similar to that of Example 2 was used to probe which of the 16 GNN sense codons could be efficiently suppressed from a pool of competing aminoacyl-tRNAs. An mRNA library was constructed containing a single random position (NNN) and encoding all 15 of the non-GNN amino acids (FIG. 7A), giving a total of 64 possible templates. The synthetic DNA template 5′-GGACAATTAC TATTTACAAC CACCATGTCA TATNNNCTGA TCTTCACCAA GTGCCGCTGG CCC-3′ (SEQ ID NO: 48; where N is T, C, A, or G) was PCR-amplified with synthetic primers T7-E (5′-AGTGAATTCT AATACGACTC ACTATAGGGA CAATTACTAT TTACAACCAC CATG-3′; SEQ ID NO: 49) and 3P (5′-UUTGTTCTGG TGGGGCCAGC GGCACTTGGT-3′; SEQ ID NO: 50). The PCR product was transcribed by T7 polymerase to generate mRNA that was ligated with T4 DNA ligase (New England Biolabs) to a flexible DNA linker containing puromycin, pF30P (5′-dA₂₁{C₉}₃dACdCP that was 5′-phosphorylated using phosphorylation reagent II (Glen Research; C₉=triethylene glycol phosphate, and P=CPG-puromycin, Glen Research) with a splint (5′-T₁₅GTTCTGGTGGGGCCA-3′; SEQ ID NO: 51). The purified fusion template was translated in rabbit reticulocyte lysate (Novagen) using standard conditions. mRNA-peptide fusion formation was stimulated by the addition of MgCl₂ and KCl to 50 mM and 0.6 M, respectively, prior to overnight incubation at −20° C.

Based on the THG73 suppressor tRNA (SEQ ID NO: 25), 16 novel tRNAs were constructed that could recognize each of the GNN codons via Watson-Crick base-pairing (see FIG. 7A; Heckler et al., Biochemistry 23:1468-1473, 1984; Robertson et al., J. Amer. Chem. Soc. 113:2722-2729, 1991). The pUC19-based plasmid harboring the gene for THG73 was mutated at the tRNA anticodon position using the QuikChange™ mutagenesis kit (Stratagene) with 16 appropriate sets of primers. Resulting clones were verified by DNA sequencing prior to synthesizing individual tRNAs in vitro from Fok I-linearized plasmids and subsequent tRNA purification by gel electrophoresis.

The 16 tRNA molecules were chemically-acylated with biocytin (see FIG. 7A). tRNAs synthesized by in vitro transcription were ligated to a molar excess of NVOC-protected biocytin-dCA with T4 RNA ligase (New England Biolabs). Reaction mixtures were extracted in an equal volume of phenol:CHCl₃:isoamyl alcohol (25:21:1, pH 5.0), and precipitated with 2.5 volumes ethanol (−20° C.). After drying, the pellets were resuspended in 1 mM sodium acetate, pH 5.2. Before adding to translation reactions, the biocytin-tRNA solution was deprotected by exposure for 5 min to a xenon lamp outfitted with a 315 nm cut-off filter.

For in vitro selection, the template library and the 16 tRNAs were added to a commercially available rabbit reticulocyte lysate (Novagen, Inc.) for translation (Jackson and Hunt, supra, 1983) to generate mRNA-peptide fusions (FIG. 7B). The mRNA-peptide fusion population contained a mixture of templates, some of which bear natural peptides and others that bear biocytin. The population of molecules bearing biocytin can be isolated on streptavidin-agarose. The mRNA-peptide fusions were initially isolated from translation reactions by dT₂₅-cellulose purification in 1 M NaCl, 100 mM Tris-HCl pH 8.0, 0.2% TRITON X-100 detergent at 4° C., washed, and eluted in ambient water. The mRNA-peptide fusions were subject to SUPERSCRIPT II RNase H⁻ reverse transcriptase (Life Technologies) before binding to streptavidin-agarose (Pierce) in 50 mM sodium phosphate pH 7.0 at 4° C. and washed in 50 mM sodium phosphate pH 8.0, 100 mM NaCl, 0.1% SDS. As such, the selection protocol includes two selective steps: 1) a competition during translation for incorporation of the unnatural residue over the endogenous pool of amino acylated tRNAs (FIG. 7B); and 2) a selection due to purification using streptavidin-agarose, which provides a quantitative means to measure the percentage of all mRNA-peptide fusions that have incorporated biocytin.

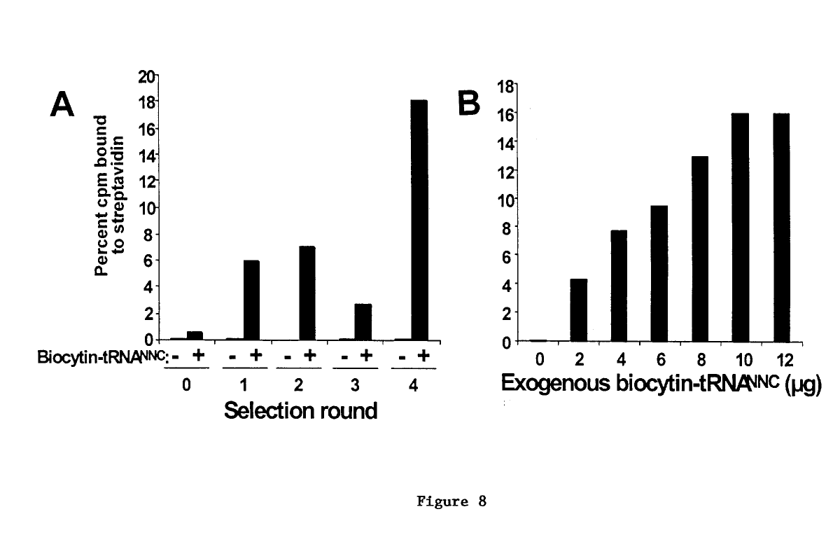

Four rounds of selection were performed on the template library. In each successive round the increase in streptavidin binding was dependent upon the presence of the biocytin-tRNA^{NNC} pool (FIG. 8A). A small but measurable amount of fusion bound streptavidin-agarose over the control reaction, which lacked template (“no template control”; FIG. 8A, round 0). The stringency of the selection was controlled by the amount of exogenous tRNAs added to the translation reaction. In rounds 0 through 2, 4.0 μg of the biocytin-tRNA^{NNC} pool was used, and selection on streptavidin produced a steady increase in the incorporation of biocytin into the library. In round 3 only 1.0 μg was used, thus increasing the stringency of the selection and resulting in an overall decrease in the amount of biocytin-bearing product generated. The benefit of this high stringency step is evident upon comparing rounds 2 and 4, as a roughly 3-fold increase in streptavidin binding occurred.

Because incorporation of biocytin should compete with the endogenous pool of aminoacyl-tRNAs, the percent of the fusion that contains biocytin should depend on the concentration of biocytin-tRNA used in the experiment. To test the concentration dependence of biocytin incorporation, the template library from round 3 was used to generate mRNA-peptide fusions in the presence of increasing amounts of the biocytin-tRNA^{NNC} pool. As shown in FIG. 8B, modest amounts of aminoacyl-tRNAs (for example, 4.0 μg) were sufficient to drive incorporation, and that lower tRNA concentrations (for example, 1.0 μg) provide increased selection stringency, consistent with observations made during the selection experiment.

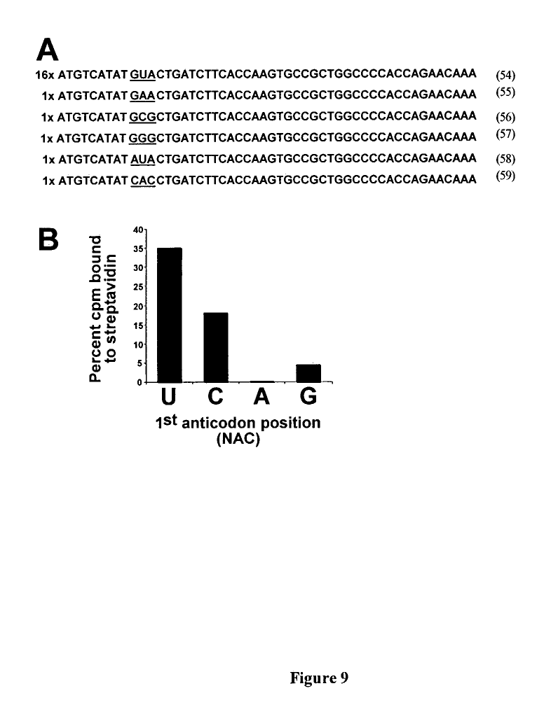

Sequence analysis of the round 4 clones revealed clear enrichment of GUA, a valine codon, at the randomized position, and no other changes to the template sequence (see FIG. 9A). Codons that occurred only once in the round 4 pool included GCG alanine, GGG glycine, GAA glutamic acid, CAC histidine, and AUA isoleucine. This result indicates that GUA codons are recognized by one of the 16 tRNAs in the mixture. the biocytin incorporation in the GUA-containing template when different chemically acylated tRNAs were used (FIG. 9B).

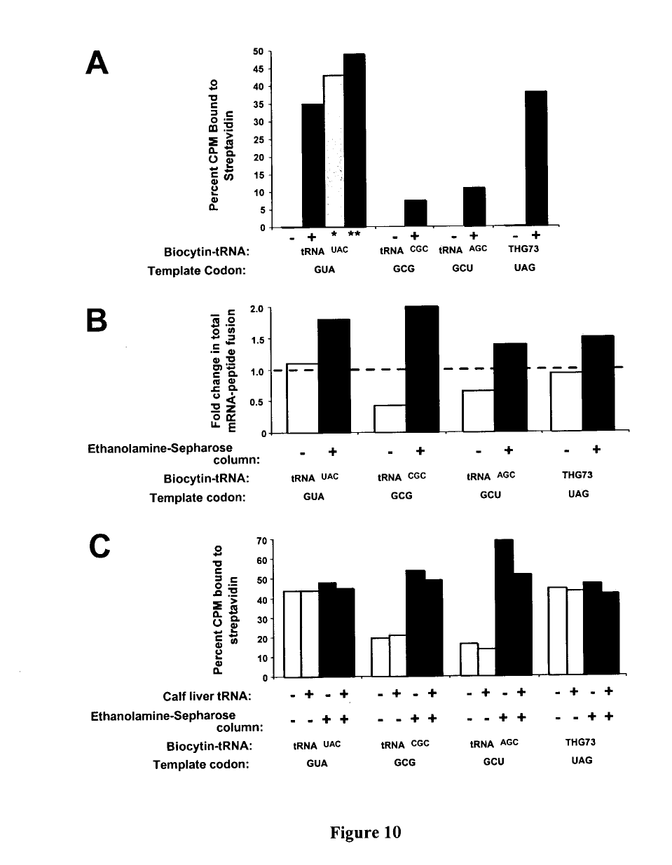

To confirm that the GUA codon was the source of biocytin incorporation, and to compare the efficiency of this reaction to UAG-based nonsense suppression, four discrete templates were synthesized containing 1) the GUA valine codon, 2) the GCG alanine codon, 3) a GCU alanine codon and 4) the UAG nonsense codon. Each of the templates was probed with a complementary biocytin-acylated THG73 variant. As shown in FIG. 10A, the efficiency of GUA-mediated sense suppression was similar to, if not better than, UAG-mediated nonsense suppression. This result is striking given that the sense suppression must compete with a natural tRNA pool (i.e., calf thymus tRNA added to the lysate to give efficient translation on many different templates) present in the lysate. Although the GCG and GCU alanine codons showed reduced incorporation relative to the GUA valine codon that were selected, the substantial incorporation present using even the GCG and GCU alanine codons nevertheless demonstrates that that supplanting natural codon pairing is not as difficult as was previously assumed.

The 16 tRNA molecule(s) responsible for inserting biocytin in response to GUA codons was determined. In principle, the GUA codon can be recognized by standard Watson-Crick pairing, via wobble interaction, or via non-canonical pairing. The GUA template was probed using four different biocytin-acylated tRNAs, each of which contained a different base in the wobble position (anticodon=UAC, CAC, AAC, or GAC). These sense suppression experiments allowed a comparison of codon:anticodon recognition from orthogonal sets of tRNAs. Binding results demonstrated that Watson-Crick recognition was by far the most efficient of the possible pairing schemes (UAC—35% of labeled peptide to streptavidin; FIG. 9B). The codon:anticodon wobble pairing of A:A proved unfavorable for GUA suppression (AAC—0.15% binding), in accordance with wobble rules (Crick, J. Mol. Biol 19:548-555, 1966). The level of GUA suppression by biocytin-tRNA^{AAC} was roughly equivalent to the background level of suppression observed for biocytin-acylated THG73 on the same template. The A:C pairing (CAC—18% binding) and A:G pairing (GAC—4.4% binding) both supported GUA suppression, though to a lesser extent than the canonical A:U interaction, consistent with a loss of one hydrogen bond in the wobble position.

GUA codons may be efficiently competed by exogenous tRNA because the corresponding tRNAs are not abundant in the lysate. If true, then generally reducing the tRNA concentration should produce a concomitant increase in sense suppression. To examine this possibility, ethanolamine-SEPHAROSE gel was used to specifically deplete tRNAs from the translation extract (Jackson et al., RNA 7:765-773, 2001, which is incorporated herein by reference). The depleted lysate, which lacks much of the endogenous tRNA population, was assayed for GUA suppression by biocytin-tRNA^{UAC}. As predicted, lowering the endogenous tRNA concentration resulted in an increase in GUA suppression. This result demonstrates that at least one reason that the GUA codon was enriched was because tRNAs complementary to this codon are not abundant in the rabbit reticulocyte lysate or the supplemented calf liver tRNA.

This result further suggests that there is likely an optimal concentration or balance of endogenous tRNAs to the sense suppressor. Lysates that completely lack tRNA do not support translation (Jackson et al., supra, 2001), whereas lysates that contain large quantities of endogenous tRNA require high concentrations of suppressor to incorporate an unnatural amino acid. As disclosed herein, the addition of 20 μg/ml calf liver tRNA, which is a lower concentration than that in the untreated, commercial lysate concentration (60 μg/ml), resulted in improved sense suppression approaching 50%. Thus, partially removing the complex endogenous tRNA population can tip the balance in favor of sense suppression by orthogonal tRNAs. This result indicates that complete elimination of competing tRNAs can allow for replacement of all of the natural amino acids, enabling synthesis of totally unnatural mRNA display libraries or polymers.

Lack of competition at GUA codons can be due to low gene dosage of this particular valine tRNA. The number of tRNA genes encoding UAC anticodons in rabbit and cow (the sources of the translation extracts) is unknown; however, in humans, only one of the 157 tRNA genes recognizes GUA via Watson-Crick pairing, whereas 11 genes code for tRNAs that recognize the GCU alanine codon. Correspondingly, sense suppression of GCU codons by biocytin-tRNA^{AGC} resulted in a 3-fold lower level of biocytin incorporation as compared to GUA suppression. Sense suppression of GCG, an alanine codon with one complementary human tRNA gene and one occurrence in the round 4 pool, gave a 5-fold lower signal than GUA-based suppression. Whereas GUA codons can be recognized only via canonical Watson-Crick pairing, GCG and GCU codons can both be recognized canonically and via wobble interactions, making them less likely to serve as blanks in the genetic code.

The GUA codon has one of the lowest uses of GNN type codons in higher eukaryotes and correspondingly, a low abundance of its isoacceptor tRNAs (Ikemura and Ozeki, Cold Spring Harbor Symp. Quant. Biol. 47:1087-1097, 1983; Kanaya et al., J. Mol. Evol. 53:290-298, 2001, each of which is incorporated herein by reference), which is likely why it is selected for sense suppression. If this is true, then reducing the overall tRNA concentration should increase suppression at arbitrarily chosen sense codons. Previously, Jackson and coworkers developed a protocol using ethanolamine-SEPHAROSE gel to specifically deplete tRNAs from a translation extract (Jackson et al., supra, 2001). In the present study, commercial rabbit reticulocyte lysate was subjected to ethanolamine-SEPHAROSE gel to create a lysate having a partially depleted endogenous tRNA population; the lysate was then assayed GUA suppression by biocytin-tRNA^{UAC} (FIG. 10A).

In the commercially available lysate, lowering the endogenous tRNA concentration produced a modest increase in GUA suppression (FIG. 10A). This result indicates that an optimal concentration or balance of endogenous tRNAs to the sense suppressor likely exists. Lysates totally lacking tRNA will not support translation (Jackson et al., supra, 2001), whereas lysates that contain large quantities of endogenous tRNA will require high concentrations of suppressor to incorporate an unnatural amino acid. As such, partially removing the complex endogenous tRNA population can tip the balance in favor of sense suppression by orthogonal tRNAs and, therefore, a more stringent elimination of competing tRNAs can allow for replacement of all the natural amino acids, enabling synthesis of trillion-member unnatural mRNA display libraries.

In an effort to determine the impact of endogenous tRNA depletion in translation reactions on sense codon suppression, an “in-house” rabbit reticulocyte lysate was used. In-house preparations of rabbit reticulocyte lysate were prepared as previously described (Jackson and Hunt, supra, 1983). Briefly, 10 mL of completely clarified rabbit reticulocyte lysate was incubated for 30 min at ambient temperature with 0.2 mL 1.0 mM hemin (Sigma) in 85% ethylene glycol, 0.1 mL 5 mg/mL creatine kinase solution (50% v/v aqueous glycerol; Boehringer Mannheim), 0.1 mL 0.1 M CaCl₂ and 0.1 mL micrococcal nuclease (15,000 units/mL; Boehringer Mannheim). The reaction was quenched by the addition of 0.1 mL 0.2 M EGTA-KOH, pH 7.5, and placing the solution on ice. Salts were added to a concentration of 50 mM KCl and 0.25 mM MgCl₂, and then 2 mL of this material was passed over an ethanolamine-Sepharose column (0.5-mL bed volume; 0.4-cm inner diameter×10-cm height) pre-equilibrated in 5 mL buffer A with additional 50 mM KCl and 0.25 mM MgCl₂ (Jackson et al., 2001). The first 0.5 mL fraction of lysate that passed through the column was discarded and the subsequent 4×0.5 mL fractions were pooled and saved in 250 μL aliquots. Rabbit reticulocyte lysates depleted of endogenous tRNA were either used immediately for translation reactions or snap-frozen in an ethanol-dry ice bath and stored at −80° C.

For chromatographic depletion of endogenous tRNAs for sense suppression, ethanolamine-SEPHAROSE gel resin was prepared from epoxy-activated SEPHAROSE 6B gel (Sigma) as previously described (Jackson et al., supra, 2001). This resin was used to deplete endogenous tRNA in commercial and in-house preparations of rabbit reticulocyte lysate. All procedures were performed at 4° C. unless otherwise specified. For tRNA depletion in commercial translation lysates, 100 μL of a 50% ethanolamine-SEPHAROSE gel slurry in buffer A (25 mM KCl, 10 mM NaCl, 1.1 mM MgCl₂, 0.1 mM EDTA, 10 mM HEPES-KOH, pH 7.2) with additional 50 mM KCl and 0.25 mM MgCl₂ was incubated for 45 min with rabbit reticulocyte lysate (Novagen, Inc.) prior to brief centrifugation at 1500×g to clarify the desired supernatant.

The in-house rabbit reticulocyte lysate was depleted of endogenous tRNA by passage over the ethanolamine-SEPHAROSE gel column. The level of endogenous tRNA depletion was assessed by the fold change in total mRNA-peptide fusion ((³⁵S)-labeled material eluted from dT₂₅-cellulose) upon the addition of 1.5 μg calf liver tRNA (FIG. 10B). Prior to passing lysate through the column translation appeared to be independent of calf liver tRNA for GUA and UAG templates, and in the cases for GCG and GCU templates the amount of mRNA-peptide fusion formed was actually inhibited by added tRNA. This inhibitory effect was reduced upon pre-incubation (5-20 min) of the calf liver tRNA in the translation reaction before adding templates, indicating that the calf liver tRNA is in large part deacylated, and that GCG and GCU isoacceptor tRNAs from this pool exert a larger effect than those for GUA due to differences in concentrations. Once passed through the ethanolamine-SEPHAROSE gel column, the translation lysate exhibited a tRNA-dependence to form mRNA-peptide fusions for all templates tested (FIG. 10B), thus demonstrating the efficiency with which the chromatographic step removes endogenous tRNAs. It should be noted that a complete elimination of endogenous tRNAs was not observed, suggesting that the background counts measured by Jackson et al. for tRNA-dependent lysate without tRNA supplementation (Jackson et al., supra, 2001) can still support efficient translation of mRNA-peptide fusions. 101661 Biocytin incorporation was measured at sense and nonsense codons to compare rabbit reticulocyte lysate before and after ethanolamine-SEPHAROSE gel treatment to remove tRNA. Before removal of tRNAs from the in-house lysate, the four templates gave biocytin incorporation levels (FIG. 10C) similar to those detected using the commercial lysate (FIG. 10A). However, suppression of the GCG and GCU codons increased dramatically when tRNA was removed from the in-house lysate preparation. This tRNA depleted lysate thus gave comparable suppression of GUA, GCG, GCU and UAG codons (FIG. 10C). Suppression at the amber and GUA codons appeared to be independent of tRNA depletion. This observation is in line with the conclusion that the GUA codon was selected for biocytin incorporation because its isoacceptor tRNA concentrations in the lysate (both commercial and in-house preparations) are very low. The marked improvement observed at GCG and GCU codons clearly demonstrates that removing endogenous and competing tRNAs produces blanks in the genetic code for sense suppression at a variety of sense codons. Further, this result demonstrates that controlling the tRNA concentration provides a general means to reprogram arbitrarily chosen sense codons.

In focusing on the GNN quadrant of the universal genetic code, a selection experiment was designed using mRNA display to identify sense codons that can be efficiently suppressed. This same strategy can be used to identify codons in the other three quadrants (UNN, CNN, and ANN codons) that can be easily suppressed in the rabbit reticulocyte or other in vitro translation system. As disclosed herein, sense suppression efficiency depends on the concentration of both the endogenous tRNA pool and the suppressor tRNA used. As such, the elimination of competing tRNAs from the translation lysate can create completely suppressible codons—synthetic blanks in the genetic code that can permit high efficiency insertion of unnatural amino acids into mRNA display libraries. Such easily suppressed sense codons will likely vary depending on the translation system or organism used, and suppression efficiency is expected to be inversely proportional to tRNA abundance. As such, translation systems that are partially or totally reconstituted (Forster et al., Anal. Biochem. 297:60-70, 2001; Shimizu et al., Nat. Biotechnol. 19:751-755, 2001), or where tRNA concentrations (Jackson et al., supra, 2001) or synthetase activity (Tao and Schimmel, Expert Opin. Investig. Drugs 9:1767-1775, 2000) can be controlled, provide useful platforms for rewriting large blocks of the universal code, thus enabling the creation of trillion member or more unnatural display libraries.

### EXAMPLE 4

### SYNTHESIS OF N-METHYL-PHENYLALANINE OLIGOMERS USING SENSE SUPPRESSION OF GUA CODONS

This Example demonstrates oligomers of unnatural amino acids can be assembled as RNA-peptide fusions using templates containing sense codons.

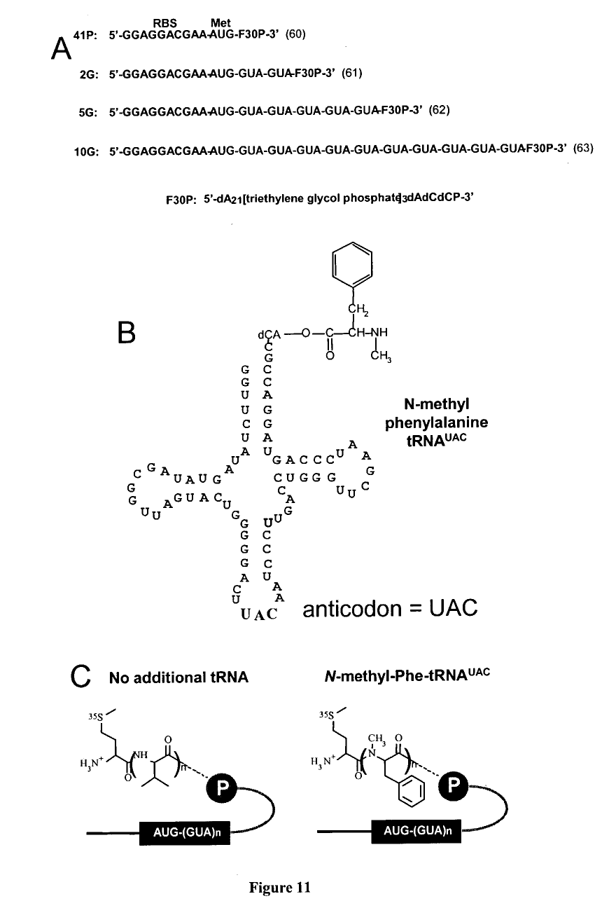

The results in Example 3 demonstrated that the unnatural residue biocytin can be incorporated efficiently at GUA codons in a commercially available translation extract, and further demonstrated that biocytin can be incorporated efficiently at GCG and GCU codons in a modified (in-house) translation extract. Because the GUA codon enabled efficient incorporation of the unnatural amino acid biocytin, experiments were designed to confirm that an unnatural oligomer could be constructed using mRNA display on GUA-containing templates. Three templates (2G, 5G, and 10G; SEQ ID NOS: 61 to 63, respectively) and one size control (41P; SEQ ID NO: 60) were constructed (see FIG. 11A) to examine oligomer synthesis. The 2G, 5G, and 10G templates contain 2, 5, and 10 tandem GUA codons respectively.

N-methyl-phenylalanine was chosen as the monomer to examine. N-substituted monomers may result in oligomers that have advantages over natural amino acids in terms of their stability to proteolysis (Miller et al., Drug Devel. Res. 35:20-32, 1995), their ability to bind certain proteins (Nguyen et al., Science 282:2088-91, 1998), or their pharmacological properties. Additionally, N-substituted residues have been shown to be capable of participating in protein synthesis with good efficiency (Bain et al., Tetrahedron, 47:2389, 1991; Cornish et al., Angew. Chem. Int. Ed. Engl. 34:621-633, 1995, each of which is incorporated herein by reference).

Based on the GUA suppressor variant set forth in Example 3, a GUA suppressor tRNA aminoacylated with N-methyl-phenylalanine was prepared (FIG. 11B). For synthesis of N-methyl, N-nitroveratrylcarbonyl phenylalanine cyanomethyl ester, 50 mg N-methyl-phenylalanine (280 μmol) was dissolved in 1 mL 10% NaCO₃+500 μL dioxane and cooled to 4° C. in an ice bath. To this, 77 mg 4,5-dimethoxy-2-nitrobenyl chloroformate was added in 1.6 mL dioxane:THF (1:1). The reaction was stirred at 0° C. for 1 hr, then 25 C. for 3 hr. The reaction mixture was poured into 15 mL water and extracted twice with 5 mL diethyl ether. The aqueous layer was acidified with concentrated HCl (pH=2) and then extracted twice with ethyl acetate. The organic layer was dried over MgSO₄ and concentrated by rotary evaporation. The crude product was dissolved in 1.5 mL dry DMF and 1.5 mL chloracetonitrile. 1 mL (24 equivalents) TEA was added and the reaction was stirred under nitrogen overnight at 25° C. Unreacted chloracetonitrile and solvent were removed by rotary evaporation and the product was purified by Silica gel chromatography in 95:5 CH₂Cl₂: MeOH. (R_{f}=0.7). The overall stepwise yield was approximately 47%. Analysis by ESI-MS resulted in an observed {M+Na}⁺ peak of 480.2 compared with the expected product {M+Na}⁺=480.4.

For synthesis of N-methyl, N-nitroveratrylcarbonyl phenylalanine-dCA, commercially synthesized pdCpA was dissolved in 0.01 M tetrabutylammonium hydroxide and allowed to stand at 25° C. for 4 hr, then lyophilized to dryness. 4 μmol of lyophilized pdCpA was transferred to a dry round bottom flask and 60 mg (131 μmol) N-methyl, N-nitroveratrylcarbonyl phenylalanine cyanomethyl ester was added. The solid reagents were dissolved in 400 μL dry DMF and a catalytic amount of tetrabutylammonium acetate was added. The reaction was stirred under nitrogen for 5 hr. The final product was purified by C18 semi-preparative HPLC. Solvent A=25 mM NH₄OAC (pH=4.5): CH₃CN (95:5). Solvent B=25 mM NH₄OAC (pH=4.5): CH₃CN (10:90). Gradient=0-100% B in 60 min. Flow=5 mL/min. The product retention time was approximately 22 min. The fractions containing the product were lyophilized to dryness. The lyophilized product was redisolved in 10 mL of 10 mM Acetic acid and lyophilized again. The final product was quantitated by A₃₅₀. Analysis by ESI-MS resulted in an observed {M−H}⁻=1035.4, compared with the expected {M−H)⁻=1035.2.

Ligation of NVOC-protected N-methyl-phenylalanine-dCA to purified tRNA^{UAC} was performed as follows. Purified tRNA^{UAC} was ligated to a molar excess of NVOC-protected N-methyl-phenylalanine-dCA with T4 RNA ligase (New England Biolabs). The reaction mixture was extracted in an equal volume of phenol:CHCl₃:isoamyl alcohol (25:21:1, pH 5.0), and precipitated with 2.5 volumes ethanol (−20° C.). After drying, the pellet was resuspended in 1.0 mM sodium acetate, pH 5.2 and adjusted to 1.0 mg/mL for each acylated tRNA. Before adding to translation reactions, N-methyl-phenylalanine-tRNA^{UAC} was deprotected by a xenon lamp outfitted with a 315-nm cut-off filter for 5 min to remove the NVOC group.

Programming a translation with (GUA)_{N} templates (FIG. 11A) and the N-methyl-phenylalanine tRNA (FIG. 11B) should result in mRNA-peptide fusions containing N-methyl-phenylalanine oligomers (FIG. 11C). Translation reactions where no chemically acylated tRNA is added, should result in mRNA-peptide fusions containing valine oligomers or no product at all (FIG. 11C). Generation and analysis of mRNA-peptide fusions on 41P, 2G, 5G and 10P templates. Synthetic RNA hybrid templates (FIG. 11A) included 41P, (5′-GGAGGACGAA AUG-F30P-3′; SEQ ID NO: 60); 2G, (5′-GGAGGACGAA AUGGUAGUA-F30P-3′; SEQ ID NO: 61); 5G, (5′-GGAGGACGAA AUGGUAGUAG UAGUAGUA-F30P-3′; SEQ ID NO: 62); and 10G, (5′-GGAGGACGAA AUGGUAGUAG UAGUAGUAGU AGUAGUAGUA GUA-F30P-3′; SEQ ID NO: 63), where F30P is 5′-dA₂₁{C₉}₃dAdCdCP; C₉=triethylene glycol phosphate, and P=CPG-puromycin (Glen Research); F30P serves as a flexible DNA linker (see FIGS. 11A and 11C). Templates were gel-purified and desalted by ethanol precipitation, and 10 pmol of material was translated in tRNA-depleted rabbit reticulocyte lysate using standard conditions (30° C. for 60 min) in the presence or absence of 2 μg N-methyl-phenylalanine-tRNA^{UAC}. Amino acid supplementation in the translation reaction was limited to (³⁵S)-methionine only. RNA-peptide fusion formation was stimulated by the addition of MgCl₂ and KCl to 50 mM and 0.6 M, respectively, prior to overnight incubation at −20° C.

The RNA-peptide fusions were initially isolated from translation reactions by dT₂₅-cellulose binding in 5 mL isolation buffer (1M NaCl, 100 mM Tris-HCl pH 8.0, 0.2% TRITON X-100 detergent) at 4° C. for 45 min, washed in 700 μL isolation buffer seven times at 4° C., and eluted in water (ambient temperature). Purified RNA-peptide fusions were concentrated via ethanol precipitation in the presence of 30 μg linear acrylamide (Ambion) and resuspended in 40 μL TE buffer (10 mM Tris-HCl, pH 8.0, 1 mM EDTA).

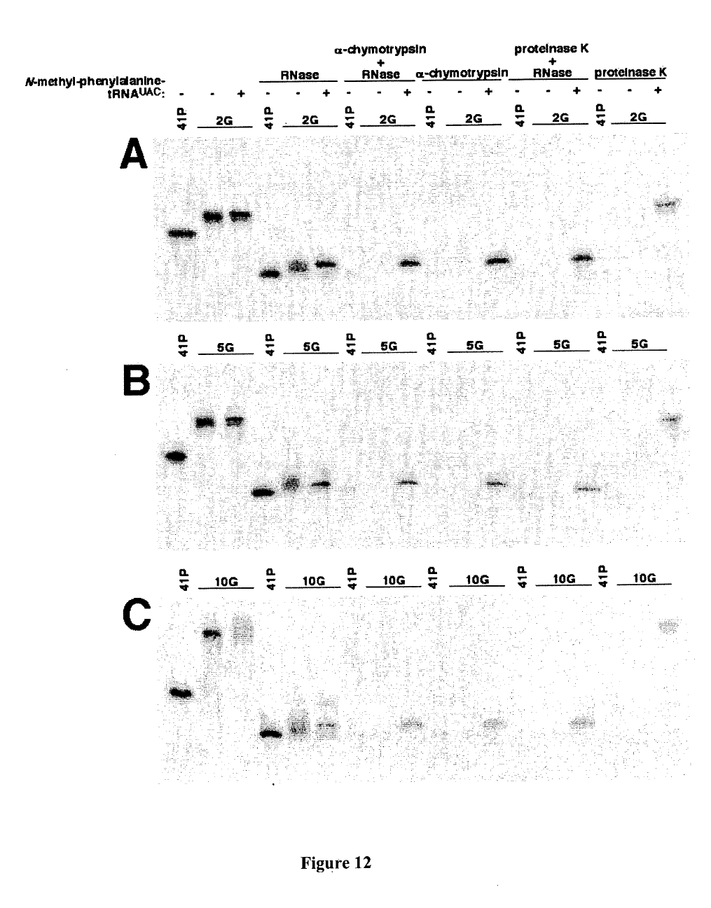

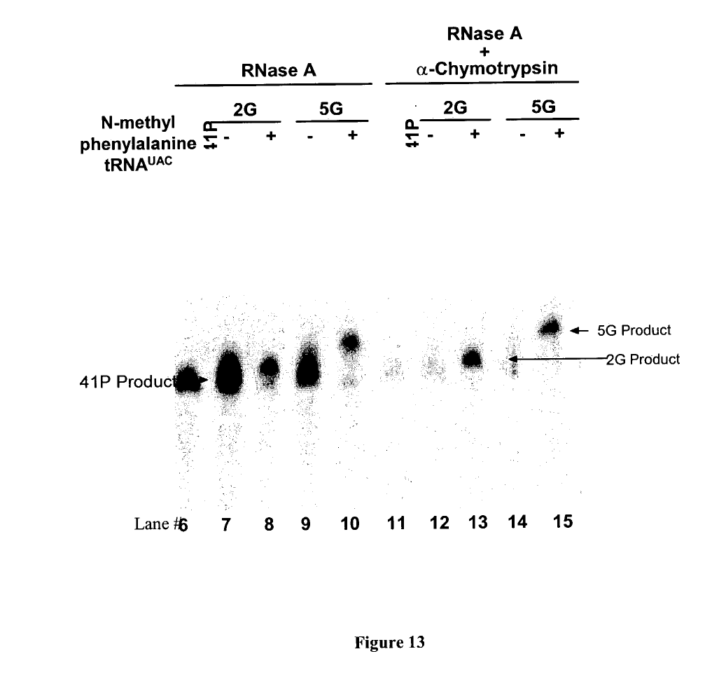

The proteolytic and nuclease susceptibility of 41P, 2G, 5G and 10P template products was tested to examine incorporation of the N-methyl-phenylalanine. (³⁵S)-met labeled mRNA-peptide fusions (10 μL) were treated with either 1 μg RNase A (DNase-free; Qiagen) at ambient temperature, 10 μg α-chymotrypsin for 90 min at 25° C., a combination of RNase A and α-chymotrypsin, or 10 μg proteinase K for 30 min at 37° C. The results of these reactions were analyzed using 12.5% urea-PAGE gel (FIG. 13), or 10% SDS tricine gel electrophoresis (FIG. 12) (Schagger and Jagow, Anal. Biochem. 166:368, 1987, which is incorporated herein by reference).

Translation of the 2G, 5G, and 10G templates resulted in RNA-peptide fusion formation both in the presence and absence of chemically acylated N-methyl-phenylalanine tRNA as judged by (³⁵S) methionine incorporation assayed on tricine SDS-PAGE (FIGS. 12A to 12C). The radiolabeled product was attached to the linker portion of the molecule as judged by RNase A treatment, which removes the template RNA nucleotides, but does not cleave the linker or peptides (FIG. 12; RNase lanes).

Addition of the N-methyl-phenylalanine-acylated suppressor tRNA resulted in the generation of radiolabeled product that was resistant to either chymotrypsin or proteinase K treatment for the 2G, 5G, and 10G templates. The resulting radiolabeled material was consistent with product formation of N-methyl-phenylalanine oligomers as RNA-peptide fusions. No chymotrypsin-resistant or proteinase K-resistant product was detected if the N-methyl-phenylalanine tRNA was omitted, consistent with synthesis of a Met-(Val)_{N} mRNA peptide fusion product in the absence of chemically-acylated suppressor tRNA^{UAC}.

Treatment with chymotrypsin alone produced a product that migrated at the same mobility as the product of chymotrypsin+RNase A, consistent with the idea that the chymotrypsin enzyme was contaminated with RNase activity. Proteinase K treated lanes showed decreasing mobility in the series 41P, 2G, 5G, and 10G, likely due to the length of mRNA present in the product.

Urea-PAGE analysis of the 2G and 5G templates indicated that both run with lower mobility than the singly modified 41P template (FIG. 13; lanes 13 and 15). Since the RNase-resistant portion of each template is the same (F30P), this analysis reveals that a longer chymotrypsin-resistant peptide was attached to 5G than to 2G, and that the 2G peptide was longer than the single amino acid attached to the 41P template. This same analysis was used to examine the 10G template. RNase and chymotrypsin treatment of the 10G template resulted in material that did not enter the urea polyacrylamide gel efficiently, consistent with the expected low water solubility of a Met-(N-methyl-phenylalanine)₁₀ peptide.

While the invention has been described in detail with reference to above example, it will be understood that modifications and variations are encompassed within the scope of the invention.

What is claimed is:

1. A method for producing a library of diverse nucleic acid-peptide fusion molecules, wherein each molecule of the library comprises an encoding nucleic acid translationally linked to an encoded peptide, and wherein at least one peptide of the library contains at least one unnatural amino acid residue, the method comprising contacting, under conditions suitable for in vitro translation of a peptide comprising at least one unnatural amino acid residue,
a) a plurality of RNA molecules comprising peptide coding sequences,
wherein each RNA molecule has a 5′ end and a 3′ end,
wherein each RNA molecule is linked at the 3′ end to a peptide acceptor, and
wherein the peptide acceptor is translationally linked to a C-terminal amino acid residue of a growing protein chain by a ribosomal peptidyl transferase, and
b) at least a first aminoacylated tRNA comprising a first unnatural amino acid residue, wherein at least one peptide coding sequence of the plurality of RNA molecules contains a codon specific for the first aminoacylated tRNA,
thereby producing a library of diverse nucleic acid-peptide fusion molecules, wherein at least one peptide of the library contains at least one unnatural amino acid residue.

2. The method of claim 1, wherein the first aminoacylated tRNA comprises a first orthogonal amino acylated tRNA.

3. The method of claim 2, wherein the first orthogonal aminoacylated tRNA is a suppressor tRNA, which is specific for a stop codon.

4. The method of claim 3, wherein the first orthogonal aminoacylated tRNA is an amber suppressor tRNA, and wherein the stop codon is UAG.

5. The method of claim 1, wherein the first aminoacylated tRNA is specific for a codon encoding an amino acid.

6. The method of claim 1, wherein the first aminoacylated tRNA comprises an unnatural amino acid that is an analog or derivative of the amino acid encoded by the codon.

7. The method of claim 1, comprising further contacting the plurality of RNA molecules with at least a second aminoacylated tRNA comprising a second unnatural amino acid residue.

8. The method of claim 2, comprising further contacting the plurality of RNA molecules with at least a second orthogonal aminoacylated tRNA comprising a second unnatural amino acid residue.

9. The method of claim 1, comprising further contacting the plurality of RNA molecules with a plurality of aminoacylated tRNA molecules, each aminoacylated tRNA molecule comprising an unnatural amino acid residue, wherein the plurality of aminoacylated tRNA molecules are specific for all of the codons of the peptide coding sequences of the plurality of RNA molecules.

10. The method of claim 1, wherein the unnatural amino acid comprises a ligand that specifically binds a target molecule.

11. The method of claim 10, wherein the ligand is a small organic molecule, a peptide, a polynucleotide.

12. The method of claim 11, wherein the small organic molecule is an antibiotic.

13. The method of claim 11, wherein the small organic molecule is a nucleoside, a nucleoside analog, a nucleotide, or a nucleotide analog.

14. The method of claim 10, wherein the target molecule is a protein or a nucleic acid molecule.

15. The method of claim 14, wherein the protein is an enzyme.

16. The method of claim 15, wherein the enzyme is a kinase or a phosphatase.

17. The method of claim 14, wherein the protein is a cellular protein of a signal transduction pathway.

18. The method of claim 14, wherein the protein is a cell surface receptor.

19. The method of claim 10, wherein the target molecule comprises a hormone or a cytokine.

20. The method of claim 1, further comprising isolating nucleic acid-peptide fusion molecules of the library, wherein at least one peptide of the nucleic acid-peptide fusion molecules contains at least one unnatural amino acid residue, thereby obtaining isolated nucleic acid-peptide fusion molecules.

21. The method of claim 20, further comprising isolating at least one peptide of the isolated nucleic acid-peptide fusion molecules, said peptide comprising at least one unnatural amino acid.

22. The method of claim 1, wherein the unnatural amino acid residue comprises a ligand for a target molecule, said method further comprising contacting nucleic acid-peptide fusion molecules of the library with the target molecule under conditions suitable for a specific interaction of the target molecule with the ligand.

23. The method of claim 22, further comprising isolating nucleic acid-peptide fusion molecules that specifically interact with the target molecule, thereby obtaining isolated nucleic acid-peptide fusion molecules comprising peptides that specifically interact with the target molecule.

24. The method of claim 22, wherein the peptides of the nucleic acid-peptide fusion molecules are comprised entirely of unnatural amino acid residues.

25. The method of claim 23, further comprising isolating at least one peptide of the isolated nucleic acid-peptide fusion molecules comprising peptides that specifically interact with the target molecule, wherein said peptide comprises at least one unnatural amino acid and specifically interacts with the target molecule.

26. The method of claim 1, further comprising contacting the plurality of RNA molecules and at least a first aminoacylated tRNA with at least one aminoacyl tRNA synthetase inhibitor.

27. The method of claim 26, wherein the aminoacyl tRNA synthetase inhibitor is an aminoacyl sulfamide.

28. A library of diverse nucleic acid-peptide fusion molecules produced by the method of claim 1, wherein each molecule of the library comprises an encoding nucleic acid translationally linked to an encoded peptide, and wherein at least one peptide of the library comprises at least one unnatural amino acid residue.

29. A population of nucleic acid-peptide fusion molecules isolated by the method of claim 20, wherein at least one peptide of the nucleic acid-peptide fusion molecules comprises at least one unnatural amino acid residue.

30. A population of peptides isolated from the nucleic acid-peptide fusion molecules of claim 29, wherein at least one peptide of the population of peptides comprises at least one unnatural amino acid residue.

31. A peptide isolated from the nucleic acid-peptide fusion molecules of claim 29, wherein the peptide comprises at least one unnatural amino acid residue.

32. A population of nucleic acid-peptide fusion molecules isolated by the method of claim 23, wherein at least one peptide of the nucleic acid-peptide fusion molecules comprises at least one unnatural amino acid residue and specifically interacts with the target molecule.

33. A population of peptides isolated from the nucleic acid-peptide fusion molecules of claim 32, wherein at least one peptide of the population of peptides comprises at least one unnatural amino acid residue and specifically interacts with the target molecule.

34. A peptide isolated from the nucleic acid-peptide fusion molecules of claim 33, wherein the peptide comprises at least one unnatural amino acid residue and specifically interacts with the target molecule.

35. The library of claim 28, which comprises at least about 1×101³ diverse nucleic acid-peptide fusion molecules.

36. The library of claim 38, wherein the unnatural amino acid comprises an amino acid analog.

37. The library of claim 36, wherein the amino acid analog is biocytin or a peptoid monomer.

38. The library of claim 37, wherein the peptoid monomer comprises an N-substitute glycine.

39. The library of claim 38, wherein the peptoid monomer is N-methyl glycine, N—(S)-phenylethyl glycine, or N-methyl phenylalanine.

40. The library of claim 28, wherein the unnatural amino acid comprises a β-amino acid, a D-amino acid, an α-hydroxy acid, or an achiral backbone.

41. The library of claim 28, wherein the unnatural amino acid comprises a naturally occurring amino acid having a modified functional group.

42. The library of claim 41, wherein the functional group is a thiol group, an amino group, a carboxyl group, a guanidinium group, a hydroxyl group, or a phenolic group.

43. The library of claim 41, wherein the modified functional group comprises a carboxylic acid, an acid halide, a carboxylic ester, a thioester, or a carbamate.
